(19) United States
(12) Patent Application Publication
(10) Pub. No.: US 2024/0277567 A1
(43) Pub. Date: Aug. 22, 2024

(54) SYSTEM AND DEVICE INCLUDING THERMAL FLOW SENSING AND INTERNAL PRESSURE SENSING

(71) Applicant: ZOLL Medical Corporation, Chelmsford, MA (US)

(72) Inventors: Lisa M. Campana, Waltham, MA (US); Paolo Giacometti, North Grafton, MA (US); Gary A. Freeman, Waltham, MA (US); George Beck, Salem, MA (US); Dorian LeCroy, Parsippany, NJ (US)

(21) Appl. No.: 18/444,395

(22) Filed: Feb. 16, 2024

Related U.S. Application Data
(60) Provisional application No. 63/486,298, filed on Feb. 22, 2023.

(51) Int. Cl. A61H 31/00; A61M 16/00

(52) U.S. Cl. CPC A61H 31/005; A61H 31/006; A61M 16/0084; A61M 16/024; A61H 2201/107; A61H 2201/5071; A61H 2201/5082; A61H 2201/5087; A61H 2201/5089; A61H 2230/425; A61M 2016/0027; A61M 2016/0033; A61M 2205/15; A61M 2205/3368; A61M 2205/702; A61M 2230/42; A61M 2230/43

(57) ABSTRACT

A sensing device is provided for use in ventilation treatment, including a thermal mass flow sensor for measurement of gas flow inside of a gas flow conduit of the device and at least one pressure sensor for measurement of the pressure inside of the conduit. The sensing device may include at least one flow conditioner to condition of the flow of the gas inside of the conduit. The device may include an absolute pressure sensor for measurement of the pressure outside of the conduit (e.g., the ambient pressure). Systems and methods are provided that include a sensing device, or use thereof, in determining or presenting patient or treatment data or feedback, such as to a care provider. Systems and methods are provided that include determining patient airway gas flow and pressure waveforms, and that analyze morphological features of the waveforms to determine conditions of the patient or of treatment.

### BACKGROUND

Providing, and optimizing, potentially life-saving ventilation and other treatment to a patient, such as in emergency, field and pre-hospital settings, using portable equipment, poses many challenges. For example, at an emergency scene, one or several care providers may arrive using portable equipment carried to the scene, which must be quickly deployed, and one or multiple forms of treatment may be required, such as concurrently, intermittently or in rapid succession. This may include, for example, ventilation, CPR chest compressions and/or defibrillation.

For example, in bag valve mask (BVM) ventilation, a care provider may deliver breathing gas (e.g., air or oxygen supplemented gas) to a patient by repeatedly manually compressing a self-inflating bag to deliver the gas through a mask to the patient's airway and lungs. However, BVM ventilation can be difficult, and incorrect or suboptimal ventilation is common and can lead to poor outcomes or injure the patient. For example, even experienced care providers may tend to deliver breaths at too high a pressure, which can injure a patient's lungs. As an additional example, in cardiopulmonary resuscitation (CPR) chest compression treatment, incorrect or suboptimal treatment parameters (e.g., timing, rate or depth of compressions) can lead to poor outcomes or cause injury to the patient.

### SUMMARY

One example provides a sensing device for use in ventilation treatment, comprising: a gas flow conduit configured to be coupled with a patient airway; a thermal mass flow sensor, disposed at least in part inside of the conduit, configured for measurement of a flow of gas inside of the conduit; at least one pressure sensor configured for measurement of a pressure inside of the conduit, wherein the pressure inside of the conduit reflects a patient airway pressure; and at least one flow conditioner configured to condition the flow of the gas inside of the conduit; wherein the sensing device is configured to output signals for use in: determining at least one waveform associated with a value of at least one ventilatory parameter over time; and based at least in part on one or more morphological features of the at least one waveform, determine at least one current condition of the patient or at least one current condition relating to treatment of the patient.

In some examples, the pressure inside of the conduit reflects an inspiratory pressure or an expiratory pressure. In some examples, the flow of gas inside of the conduit reflects an inspiratory flow or an expiratory flow. In some examples, the at least one flow conditioner is configured to condition the flow by improving laminar flow of at least a portion of the gas inside of the conduit during inspiration. In some examples, the at least one flow conditioner is configured to improve the laminar gas flow by increasing uniformity of gas flow direction and increasing uniformity of gas flow velocity, and wherein improving of the laminar gas flow is for increasing accuracy of at least one of: flow rate measurement of the gas inside of the conduit and pressure measurement inside of the conduit. In some examples, the at least one flow conditioner comprises at least one screen. In some examples, the at least one flow conditioner comprises at least one baffle.

In some examples, the device comprises at least one absolute pressure sensor configured to sense a pressure outside of the conduit. In some examples, the device comprises at least one absolute pressure sensor configured to sense a pressure outside of the conduit, wherein the pressure outside of the conduit is an ambient pressure. In some examples, the at least one absolute pressure sensor is configured for use in calibration of the measurement of the flow of the gas inside of the conduit based on detection of an ambient pressure that is lower than a sca level ambient pressure. In some examples, the at least one absolute pressure sensor is configured for use in calibration of the measurement of the flow of the gas inside of the conduit based on detection of an ambient pressure that is lower than a sea level ambient pressure, wherein the ambient pressure is at an altitude higher than sea level.

In some examples, the at least one pressure sensor comprises: a differential pressure sensor, disposed at least in part inside of the conduit, configured to sense a pressure difference between a pressure inside of the conduit and a pressure outside of the conduit; and an absolute pressure sensor configured to sense the pressure outside of the conduit. In some examples, the differential pressure sensor and the absolute pressure sensor are configured for use in determination of the pressure inside of the conduit. In some examples, the at least one pressure sensor comprises an absolute pressure sensor configured to sense the pressure inside of the conduit. In some examples, the at least one pressure sensor comprises a second absolute pressure sensor configured to sense a pressure outside of the conduit.

In some examples, the device is configured to provide sensed flow rate data relating to the flow of the gas inside of the conduit and sensed pressure data relating to the pressure inside of the conduit for use in determining ventilation feedback and chest compression feedback. In some examples, the device is configured to provide sensed flow rate data relating to the flow of the gas inside of the conduit and sensed pressure data relating to the pressure inside of the conduit for use in determining ventilation feedback. In some examples, the device is configured for use in determining parameters comprising inspiratory flow rate, expiratory flow rate, and peak inspiratory pressure. In some examples, the device is configured to provide sensed flow rate data and sensed pressure data for use in determining bag valve mask ventilation feedback. In some examples, the device is configured to provide sensed flow rate data and sensed pressure data for use in determining bag valve mask ventilation feedback, wherein the bag valve mask ventilation feedback relates to at least one of: a breath rate, a flow rate and a volume relating to the bag valve mask ventilation.

In some examples, the device is configured to sensed flow rate data relating to the flow of the gas inside of the conduit and sensed pressure data relating to the pressure inside of the conduit for use in determining mechanical ventilation feedback. In some examples, the device is configured to provide sensed flow rate data relating to the flow of the gas inside of the conduit and sensed pressure data relating to the pressure inside of the conduit for use in determining chest compression feedback. In some examples, the device is configured to connect with at least one of: a display device, a patient monitor, a defibrillator, a portable computing device and a tablet.

In some examples, the thermal mass flow sensor comprises a hot wire anemometer. In some examples, the hot wire anemometer comprises a heater and a component configured to be heated by the heater, wherein the component is in direct contact with the gas inside of the conduit. In some examples, the device is a rigidly integrated device. In some examples, the device does not require user assembly of the device, and wherein the device does not require user connection or joining of any components of the device to each other. In some examples, the conduit, the thermal mass flow sensor, and the at least one pressure sensor are fixed relative to each other.

In some examples, the device comprises a carbon dioxide sensor for sensing carbon dioxide in the gas inside of the conduit. In some examples, the conduit, the thermal mass flow sensor, the at least one pressure sensor and the carbon dioxide sensor are fixed relative to each other. In some examples, the carbon dioxide sensor comprises a mainstream carbon dioxide sensor. In some examples, the mainstream carbon dioxide sensor is configured for use in measuring end tidal carbon dioxide. In some examples, the device comprises a connection adapter configured for facilitating electrical connection of the device to a medical device, wherein the connection adapter is rotatable relative to the conduit. In some examples, the device comprises a printed circuit board, and wherein the thermal mass flow sensor and at least a first pressure sensor of the at least one pressure sensor are coupled with the printed circuit board. In some examples, the device comprises a housing, wherein the conduit is disposed within a space at least partially enclosed by the housing.

In some examples, the measurement of the pressure inside of the conduit reflects a smaller delay, relative to a current patient airway pressure, than measurement of patient airway pressure using one or more sensors that are located at a distance from the patient's airway that is greater than a distance from the sensing device to the patient's airway. In some examples, the smaller delay causes a smaller risk of occurrence of patient ventilator asynchrony. In some examples, the smaller delay causes occurrences of patient ventilator asynchrony to be of smaller magnitudes. In some examples, the measurement of the flow of gas inside of the conduit reflects less delay, relative to a current patient airway flow, than measurement of patient airway flow using one or more sensors that are located at a distance from the patient's airway that is greater than a distance from the sensing device to the patient's airway.

In some examples, the smaller delay causes a smaller risk of occurrence of patient ventilator asynchrony. In some examples, the smaller delay causes occurrences of patient ventilator asynchrony to be of smaller magnitudes. In some examples, the at least one ventilatory parameter comprises at least one of: the flow of gas inside of the conduit and the pressure inside of the conduit. In some examples, the at least one ventilatory parameter comprises at least one of: temperature, ambient pressure, and at least one concentration of at least one gas compound. In some examples, the at least one ventilatory parameter comprises at least one concentration of at least one of: oxygen, carbon dioxide, nitrogen and nitric oxide. In some examples, the at least one current condition of the patient comprises at least one of: spontaneous breathing, Return of spontaneous circulation (ROSC) and agonal breathing. In some examples, the at least one current condition relating to treatment of the patient relates to at least one of: providing of bag valve mask (BVM) ventilation and providing of chest compressions. In some examples, the at least one current condition relating to treatment of the patient relates to at least one of: providing of chest compressions with an active compression decompression (ACD) device, providing of manual chest compressions with an ACD device, providing of automated chest compressions with an ACD device, providing of chest compressions with an impedance threshold device (ITD), providing of chest compressions with an ACD device and an ITD, providing of conventional chest compressions, and providing of mechanical chest compressions without the use of an ITD or ACD device.

In some examples, the at least one morphological condition comprises at least one negative pressure associated with a patient airway pressure waveform. In some examples, the at least one morphological condition comprises at least one flow rate associated with a patient airway flow rate waveform.

One example provides a system for use in ventilation treatment, comprising: a gas flow conduit configured to be coupled with a patient airway; a thermal mass flow sensor, disposed at least in part inside of the conduit, configured for measurement of a flow of gas inside of the conduit; at least one pressure sensor configured for measurement of a pressure inside of the conduit, wherein the pressure inside of the conduit reflects a patient airway pressure; and at least one processor configured to: receive signals from the thermal mass flow sensor and the at least one pressure sensor; and using the received signals, determine a flow rate of the gas inside of the conduit and a pressure inside of the conduit.

In some examples, the pressure inside of the conduit reflects an inspiratory pressure or an expiratory pressure. In some examples, the system comprises at least one flow conditioner configured to condition the flow of the gas inside of the conduit. In some examples, the system the system comprises at least one absolute pressure sensor, and wherein the at least one processor is further configured to, using signals received from the at least one absolute pressure sensor, determine a pressure outside of the conduit. In some examples, the at least one absolute pressure sensor is configured for use in calibration of the measurement of the flow of the gas inside of the conduit based on detection of an ambient pressure that is lower than a sea level ambient pressure.

In some examples, the at least one pressure sensor comprises: a differential pressure sensor, disposed at least in part inside of the conduit, configured to sense a pressure difference between a pressure inside of the conduit and a pressure outside of the conduit; and an absolute pressure sensor configured to sense the pressure outside of the conduit. In some examples, the differential pressure sensor and the absolute pressure sensor are configured for use in determination of the pressure inside of the conduit. In some examples, the at least one pressure sensor comprises an absolute pressure sensor configured to sense the pressure inside of the conduit. In some examples, the at least one pressure sensor comprises a second absolute pressure sensor configured to sense a pressure outside of the conduit.

In some examples, the at least one processor is configured to use sensed flow rate data relating to the flow of the gas inside of the conduit and sensed pressure data relating to the pressure inside of the conduit in determining ventilation feedback and chest compression feedback. In some examples, the at least one processor is configured to use sensed flow rate data relating to the flow of the gas inside of the conduit and sensed pressure data relating to the pressure inside of the conduit in determining ventilation feedback. In some examples, the at least one processor is configured to determine parameters comprising inspiratory flow rate, expiratory flow rate and peak inspiratory pressure. In some examples, the at least one processor is configured to use sensed flow rate data relating to the flow of the gas inside of the conduit and sensed pressure data relating to the pressure inside of the conduit in determining bag valve mask ventilation feedback to be presented to a care provider.

In some examples, the at least one processor is configured to use sensed flow rate data relating to the flow of the gas inside of the conduit and sensed pressure data relating to the pressure inside of the conduit in determining bag valve mask ventilation feedback to be presented to a care provider, wherein the bag valve mask ventilation feedback relates to at least one of: a breath rate, a flow rate and a volume relating to the bag valve mask ventilation. In some examples, the at least one processor is configured to use sensed flow rate data relating to the flow of the gas inside of the conduit and sensed pressure data relating to the pressure inside of the conduit in determining mechanical ventilation feedback to be presented to a care provider. In some examples, the at least one processor is configured to use sensed flow rate data relating to the flow of the gas inside of the conduit and sensed pressure data relating to the pressure inside of the conduit in determining feedback relating to providing of CPR chest compressions to be presented to a care provider.

In some examples, the at least one computerized device comprises the at least one processor, and wherein the at least one computerized device comprises at least one of: a display device, a patient monitor, a defibrillator, a portable computing device and a tablet. In some examples, the thermal mass flow sensor comprises a hot wire anemometer. In some examples, the hot wire anemometer comprises a heater and a component configured to be heated by the heater, wherein the component is in direct contact with the gas inside of the conduit. In some examples, the conduit, the thermal mass flow sensor, and the at least one pressure sensor are fixed relative to each other.

In some examples, the system comprises a carbon dioxide sensor for sensing carbon dioxide in the gas inside of the conduit. In some examples, the conduit, the thermal mass flow sensor, the at least one pressure sensor and the carbon dioxide sensor are fixed relative to each other. In some examples, the carbon dioxide sensor comprises a mainstream carbon dioxide sensor. In some examples, the mainstream carbon dioxide sensor is configured for use in measuring end tidal carbon dioxide. In some examples, the system comprises a connection adapter configured for facilitating electrical connection of the device to a medical device, wherein the connection adapter is rotatable relative to the conduit. In some examples, the system comprises a printed circuit board, and wherein the thermal mass flow sensor and at least a first pressure sensor of the at least one pressure sensor are coupled with the printed circuit board. In some examples, the system comprises a housing, wherein the conduit is disposed within a space at least partially enclosed by the housing.

In some examples, wherein the at least one processor is configured to, based at least in part on the determined flow rate of the gas inside of the conduit, detect a leak in the conduit. In some examples, the at least one processor is configured to: based at least in part on the determined pressure inside of the conduit, determine a patient airway pressure waveform reflecting the patient airway pressure over time; and based at least in part on one or more morphological features of the patient airway pressure waveform, determine at least one current condition of the patient. In some examples, the at least one current condition of the patient comprises spontaneous breathing. In some examples, the at least one current condition of the patient comprises ROSC. In some examples, the at least one current condition of the patient comprises agonal breathing. In some examples, the one or more morphological features comprise at least one of: a slope, a rise time, a fall time, a hold time, a plateau, a peak, a leading edge and a trailing edge.

In some examples, the at least one processor is configured to: based at least in part on the determined flow of gas inside of the conduit, determine a flow waveform reflecting the flow over time; and based at least in part on one or more morphological features of the flow waveform, determine the at least one current condition of the patient. In some examples, the at least one processor is configured to: based at least in part on the determined pressure inside of the conduit, determine a patient airway pressure waveform reflecting patient airway pressure over time; and based at least in part on one or more morphological features of the waveform, determine at least one current condition relating to treatment.

In some examples, the at least one current condition relating to treatment comprises at least one of: providing of BVM ventilation, providing of mechanical ventilation, and providing of chest compressions. In some examples, the at least one current condition relating to treatment comprises concurrent providing of BVM ventilation and chest compressions. In some examples, the one or more morphological features comprise at least one of: a slope, a rise time, a fall time, a hold time, a plateau, a peak, a leading edge and a trailing edge. In some examples, the at least one processor is configured to: based at least in part on the determined flow of gas inside of the conduit, determine a flow waveform reflecting the flow over time; and based at least in part on one or more morphological features of the flow waveform, determine the at least one current condition relating to treatment.

In some examples, the measurement of the pressure inside of the conduit reflects a smaller delay, relative to a current patient airway pressure, than measurement of patient airway pressure using one or more sensors that are located at a distance from the patient's airway that is greater than a distance from the sensing device to the patient's airway. In some examples, the smaller delay causes a smaller risk of occurrence of patient ventilator asynchrony. In some examples, the smaller delay causes occurrences of patient ventilator asynchrony to be of smaller magnitudes. In some examples, the measurement of the flow of gas inside of the conduit reflects less delay, relative to a current patient airway flow, than measurement of patient airway flow using one or more sensors that are located at a distance from the patient's airway that is greater than a distance from the sensing device to the patient's airway. In some examples, the smaller delay causes a smaller risk of occurrence of patient ventilator asynchrony. In some examples, the smaller delay causes occurrences of patient ventilator asynchrony to be of smaller magnitudes.

In some examples, the system is configured to output signaling for use in: determining at least one waveform associated with a value of at least one ventilatory parameter over time; and based at least in part on one or more morphological features of the at least one waveform, determine at least one current condition of the patient or at least one current condition relating to treatment of the patient. In some examples, the at least one ventilatory parameter comprises at least one of: the flow of gas inside of the conduit and the pressure inside of the conduit. In some examples, the at least one ventilatory parameter comprises at least one of: temperature, ambient pressure, and at least one concentration of at least one gas compound. In some examples, the at least one ventilatory parameter comprises at least one concentration of at least one of: oxygen, carbon dioxide, nitrogen and nitric oxide.

In some examples, the at least one current condition of the patient comprises at least one of: spontaneous breathing, Return of spontaneous circulation (ROSC) and agonal breathing. In some examples, the at least one current condition relating to treatment of the patient relates to at least one of: providing of bag valve mask (BVM) ventilation and providing of chest compressions. In some examples, the at least one current condition relating to treatment of the patient relates to at least one of: providing of chest compressions with an active compression decompression (ACD) device, providing of manual chest compressions with an ACD device, providing of automated chest compressions with an ACD device, providing of chest compressions with an impedance threshold device (ITD), providing of chest compressions with an ACD device and an ITD, providing of conventional chest compressions, and providing of mechanical chest compressions without the use of an ITD or ACD device.

In some examples, the at least one morphological condition comprises at least one negative pressure associated with a patient airway pressure waveform. In some examples, the at least one morphological condition comprises at least one flow rate associated with a patient airway flow rate waveform.

One example provides a method for providing feedback to a care provider in providing treatment to a patient, the method comprising: using a thermal mass flow sensor disposed within a gas flow conduit of a sensing device used in providing ventilation treatment to the patient, the conduit being coupled with an airway of the patient, determining a flow rate of gas inside of the conduit; using at least one pressure sensor disposed at least in part in the conduit, determining a pressure inside of the conduit, wherein the pressure inside of the conduit reflects a patient airway pressure; based at least in part on the determined flow rate of the gas inside of the conduit and the determined pressure inside of the conduit, determining the feedback, comprising at least one of ventilation treatment feedback and chest compression feedback, to be provided to the care provider during the treatment of the patient; and presenting the feedback to the care provider during the treatment of the patient.

In some examples, the treatment comprises the ventilation treatment, and wherein the feedback comprises ventilation treatment feedback. In some examples, the ventilation treatment comprises bag valve mask ventilation, and wherein the ventilation treatment feedback comprises bag valve mask ventilation feedback. In some examples, the bag valve mask ventilation feedback relates to at least one of: a breath rate, a flow rate and a volume relating to the bag valve mask ventilation. In some examples, the treatment comprises chest compressions, and wherein the feedback comprises chest compression feedback. In some examples, the chest compression feedback relates to at least one of: a rate and a depth of provided chest compressions. In some examples, using the thermal flow sensor comprises using a hot wire anemometer.

In some examples, the method comprises: using at least one absolute pressure sensor of the sensing device, determining a pressure outside of the conduit; and based at least in part on the determined flow rate of the gas inside of the conduit, the determined pressure inside of the conduit, and the determined pressure outside of the conduit, determining the feedback. In some examples, presenting the feedback comprises presenting visual feedback. In some examples, presenting the feedback comprises presenting audio feedback. In some examples, the feedback comprises an instruction to provide chest compressions or an instruction to provide ventilation breaths. In some examples, the method comprises: based at least in part on the determined pressure inside of the conduit, determining a patient airway pressure waveform reflecting the patient airway pressure over time; and based at least in part on one or more morphological features of the waveform, determining at least one current condition of the patient.

In some examples, the at least one current condition of the patient comprises spontaneous breathing. In some examples, the at least one current condition of the patient comprises ROSC. In some examples, the at least one current condition of the patient comprises agonal breathing. In some examples, the one or more morphological features comprise at least one of: a slope, a rise time, a fall time, a hold time, a plateau, a peak, a leading edge and a trailing edge. In some examples, the method comprises based at least in part on the determined pressure inside of the conduit, determining a patient airway pressure waveform reflecting patient airway pressure over time; and based at least in part on one or more morphological features of the waveform, determining at least one current condition relating to treatment. In some examples, the at least one current condition relating to treatment comprises at least one of: providing of BVM ventilation, providing of mechanical ventilation, and the providing of chest compressions. In some examples, the at least one current condition relating to treatment comprises concurrent providing of the BVM ventilation and chest compressions. In some examples, the one or more morphological features comprise at least one of: a slope, a rise time, a fall time, a hold time, a plateau, a peak, a leading edge and a trailing edge.

One example provides a method for use by a care provider in providing ventilation treatment to a patient, the method comprising: the care provider providing ventilation treatment to the patient, comprising use of a sensing device, the sensing device comprising: a gas flow conduit coupled with an airway of the patient, a thermal flow sensor for use in determining a flow rate of gas inside of the conduit, and at least one pressure sensor for use in determining a pressure inside of the conduit, wherein the pressure inside of the conduit reflects a patient airway pressure; the care provider receiving ventilation treatment feedback relating to the ventilation treatment being provided to the patient, the ventilation treatment feedback being determined based at least in part on the determined flow rate of the gas inside of the conduit and the determined pressure inside of the conduit; and based at least in part on the received ventilation treatment feedback, the care provider adjusting at least one parameter of the ventilation treatment being provided to the patient.

In some examples, the ventilation treatment comprises bag valve mask ventilation, and wherein the ventilation treatment feedback comprises bag valve mask ventilation feedback. In some examples, the bag valve mask ventilation feedback relates to at least one of: a breath rate, a flow rate and a volume relating to the bag valve mask ventilation. In some examples, use of the sensing device comprising the thermal flow sensor, wherein the thermal flow sensor comprises a hot wire anemometer.

In some examples, the method comprises: the care provider providing ventilation treatment to the patient, comprising use of the sensing device, wherein the sensing device comprises at least one absolute pressure sensor for use in determining a pressure outside of the conduit; and the care provider receiving the ventilation treatment feedback, the ventilation treatment feedback being determined based at least in part on the determined flow rate of the gas inside of the conduit, the determined pressure inside of the conduit, and the determined pressure outside of the conduit. In some examples, the method comprises the care provider receiving the ventilation treatment feedback, wherein the ventilation treatment feedback comprises visual feedback. In some examples, the method comprises the care provider receiving the ventilation treatment feedback, wherein the ventilation treatment feedback comprises audio feedback.

One example provides a method for use by at least one care provider in providing treatment to a patient, the method comprising: a ventilation care provider providing bag valve mask ventilation treatment to the patient using a bag valve mask ventilation device comprising a bag, with a sensing device coupled between the bag and an airway of the patient, the sensing device comprising: a conduit coupled with an airway of the patient, a thermal flow sensor for use in determining a flow rate of gas inside of the conduit, and at least one pressure sensor for use in determining a pressure inside of the conduit, wherein the pressure inside of the conduit reflects a patient airway pressure; a chest compression treatment care provider providing chest compressions to the patient, with the sensing device coupled between the bag and the airway of the patient; athe chest compression treatment care provider receiving chest compression feedback relating to the chest compressions being provided to the patient, the chest compression feedback being determined based at least in part on the determined flow rate of the gas inside of the conduit and the determined pressure inside of the conduit; and based at least in part on the received chest compression feedback, the chest compression treatment care provider adjusting at least one parameter of the chest compressions being provided to the patient.

In some examples, the determined flow rate of the gas inside of the conduit and the determined pressure inside of the conduit are determined during the providing of the chest compressions to the patient. In some examples, the chest compression feedback relates to at least one of: a rate and a depth of provided chest compressions. In some examples, the method comprises use of the sensing device comprising the thermal flow sensor, wherein the thermal flow sensor comprises a hot wire anemometer.

### BRIEF DESCRIPTION OF THE DRAWINGS

Various aspects of embodiments of the present disclosure are discussed below with reference to the accompanying figures, which are not intended to be drawn to scale. The figures are included for illustrative purposes and a further understanding of the various aspects and examples. The figures are incorporated in and constitute a part of this specification, but are not intended to limit the scope of the disclosure. In the figures, identical or nearly identical components that are illustrated in various figures may be represented by like numerals. For purposes of clarity, not every component may be labeled in every figure.

FIG. 1 illustrates an example emergency care environment including BVM ventilation with use of a flow sensing device.

FIG. 2. Illustrates portions of a simplified example of a display including BVM ventilation and CPR feedback determined using measurements for which a sensing device was used.

FIG. 3 is a block diagram illustrating an example of use of a sensing device in ventilation and chest compression treatment.

FIG. 4A illustrates simplified example patient airway pressure waveforms associated with the providing of BVM ventilation, chest compressions, and both BVM ventilations and chest compressions (against protocol).

FIG. 4B is a simplified illustration of the providing of chest compressions including use of an impedance threshold device (ITD).

FIG. 4C illustrates simplified example patient airway pressure waveforms associated with the providing of conventional chest compressions, chest compressions including use of an ITD, and chest compressions with use of an ITD and an active compression decompression (ACD) device.

FIG. 4D illustrates a simplified example patient airway pressure waveform associated with agonal breathing.

FIG. 5A illustrates an example patient airway pressure waveform associated with BVM ventilation breaths alternated with chest compressions, which waveform may be generated and used based on use of a sensing device.

FIG. 5B illustrates an example patient airway pressure waveform associated with spontaneous breathing, which waveform may be generated and used based on use of a sensing device.

FIG. 6 illustrates a comparison, in mechanical ventilation, of patient airway pressure sensing using a sensing device relative to patient airway pressure sensing at a ventilator or other coupled device.

FIG. 7 is a cross-sectional view illustrating an example sensing device including a hot wire anemometer based flow sensor, differential pressure sensing to sense internal pressure, and absolute external pressure sensing.

FIG. 8 is a cross-sectional view illustrating an example sensing device including hot wire anemometer based flow sensing, hot wire anemometer based differential pressure sensing to sense internal pressure, and absolute external pressure sensing.

FIG. 9 is a cross-sectional view illustrating an example sensing device including hot wire anemometer based flow sensing and absolute internal pressure sensing.

FIGS. 10A and 10B are cross-sectional views illustrating example sensing devices including hot wire anemometer based flow sensing, absolute internal sensing and an added flow conditioner.

FIG. 11 is a cross-sectional view illustrating an example sensing device including hot wire anemometer based flow sensing, absolute internal pressure sensing, and a flow conditioner that is an integrated part of the housing of the device.

FIG. 12 includes views illustrating several different types of flow conditioners.

FIGS. 13A-C include a perspective view, an inside view and a cut-out view illustrating a sensing device including integrated CO2 sensing and using hot wire anemometer flow sensing.

FIG. 14 includes top and bottom perspective views illustrating a CO2 sensor connection adapter for a sensing device including integrated CO2 sensing.

FIG. 15A-B include perspective and exploded views illustrating a sensing device including a rotatable connection adapter with clasp attachment.

FIGS. 16A-B include perspective and exploded views illustrating a sensing device including a rotatable connection adapter with magnetic attachment.

FIG. 17 depicts plots that show absolute pressure and flow rate for a spontaneously breathing patient over time, based on measurements using a sensing device.

FIG. 18 depicts plots that show absolute pressure and flow rate over time for a patient receiving positive pressure ventilation with a mechanical ventilator over time, based on measurements using a sensing device.

FIG. 19 depicts plots that show absolute pressure and flow rate over time for a patient receiving alternating BVM ventilation breaths and chest compressions, based on measurements using a sensing device.

FIGS. 20A-C depict example display dashboards for providing feedback based on measurements using a sensing device.

FIG. 21 depicts an example ventilation timer display based on measurements using a sensing device.

FIG. 22A-B depict example plots showing expired CO2 tension versus exhaled volume based on measurements using a sensing device.

FIG. 23 depicts an example plot of pressure versus volume during BVM ventilation based on measurements using a sensing device.

FIG. 24 is a block diagram illustrating example components of various devices described with reference to preceding figures.

### DETAILED DESCRIPTION

Some embodiments provide devices, systems, apparatuses, methods and computer readable media for use in treatment of a patient, such as BVM ventilation treatment or CPR chest compression treatment, as well as for use in assessment of conditions relating to treatment or to the patient.

The sensing device may include a gas flow conduit coupled with the airway of a patient, to allow the flow of gas to and from the patient (e.g., during inspiration and expiration). The sensing device may further include a thermal mass flow sensor, such as a hot wire anemometer, placed at least in part inside of the conduit, for measurement of the flow of gas (e.g., flow rate) inside of (e.g., in the lumen of) the conduit. The sensing device may further include one or more pressure sensors for measurement of a pressure inside of the conduit (which can include, e.g., peak inspiratory pressure (PIP), such as inspiratory pressure or expiratory pressure. The sensed flow rate and pressure inside of the conduit may reflect, and be used to determine or represent, patient airway gas flow rate and pressure, e.g., during inspiration and expiration. The thermal mass flow sensor may allow for highly sensitive and accurate flow rate measurements, even at low flow rates. The sensing device may include one or more electrical circuit boards or printed circuit boards (PCBs) to which the sensors may be coupled.

Some embodiments provide a single, integrated sensing device, such as for use in pre-hospital settings (including any non-hospital setting, whether or not hospital care follows), and such as for use with portable equipment. The sensing device can provide for patient airway flow rate measurements (since the device is coupled with the patient's airway), including inspiratory and expiratory flow rates, and associated volume measurements, including tidal volume. The sensing device can further provide for patient airway pressure measurements, including peak inspiratory pressure (PIP).

Various features of embodiments of flow sensors and systems as described herein may incorporate or utilize features described in U.S. Pat. No. 11,433,211, issued on Sep. 6, 2022 and entitled, “Flow Sensor for Ventilation,” which is hereby incorporated herein by reference in its entirety.

In various embodiments, various pressure sensor configurations may be used in measurement of the pressure inside of the gas flow conduit of a sensing device (e.g., inspiratory pressure or expiratory pressure), as well as the pressure outside of the conduit (e.g., the ambient or environmental pressure). In some embodiments, the pressure inside of the conduit may be measured using a differential pressure sensor in measurement of a pressure difference between the inside and outside of the conduit, along with an absolute pressure sensor to measure the pressure outside of the conduit. In some embodiments, absolute pressure inside of the conduit may be measured using an absolute pressure sensor. However, in some such embodiments, an absolute pressure sensor to measure the pressure outside of the conduit may also be included (such as to confirm or enhance internal pressure measurement, or to facilitate correcting for altitude, as described further herein). Various features of embodiments of pressure sensing, pressure sensors, and pressure sensing, as described herein, may incorporate or utilize features described in previously incorporated by reference U.S. Pat. No. 11,433,211, issued on Sep. 6, 2022 and entitled, “Flow Sensor for Ventilation.”

In some embodiments, a sensing device may include one or more flow conditioners, such as may include one or more flow restrictors, baffles or screens. A flow conditioner be used, for example, to condition the flow, such as by increasing laminar flow of the inspiratory and/or expiratory gas flow, or to increase turbulence of flow to remove biases from jetting flow profiles, such as to increase the accuracy of flow and pressure sensing. For example, jetting may result in cross-sectional areas of the conduit of a sensing device that have uneven flow profile distributions, which can lead to a measurement that is not accurate relative to the entire cross-section. In various embodiments, a flow conditioner may be separate from a housing of the sensing device or may be integrated with or formed as part of the housing.

In some embodiments, a sensing device may include an integrated carbon dioxide (CO2) sensor, such as a mainstream CO2 sensor, such as may be used for measurement of EtCO2 or determination of a capnographic waveform Various features of embodiments of CO2 sensing and a CO2 sensor, as described herein, may incorporate or utilize aspects of features described in previously incorporated by reference U.S. Pat. No. 11,433,211, issued on Sep. 6, 2022 and entitled, “Flow Sensor for Ventilation.”

In some embodiments, a sensing device may include a rotatable, such as 360 degree rotatable, connection adapter, such as may be used in facilitating fast, easy and reliable wired connection of the sensing device to a coupled medical device such as a defibrillator, medical device, monitor, computing device or portable computing device such as a tablet or smart phone. Various features of embodiments of rotatable connection and a rotatable connection adapter, as described herein, may incorporate or utilize aspects of features described in previously incorporated by reference U.S. Pat. No. 11,433,211, issued on Sep. 6, 2022 and entitled, “Flow Sensor for Ventilation.”

In some embodiments, use of a sensing device for sensing in measurement of patient airway flow rates, pressures and potentially other ventilator parameters, such as CO2 (e.g., EtCO2), may provide advantages, for example, relative to such measurements made using sensing occurring at a different device connected, such as by wire or tubing, to the patient (e.g., a ventilator, defibrillator, other medical device or other computing device). For example, since sensing using a sensing device occurs extremely close to the patient's airway, whereas sensing using another connected device may occur less close to the patient's airway, use of a sensing device may allow for measurements, such as patient airway flow rate and pressure measurements, that reflect or include less time delay relative to the current patient airway flow rates or pressures that the measurements are used to reflect or represent, relative to such measurements based on sensing occurring at another connected device. As such, use of a sensing device can lead to less delayed (which can include non-delayed) and therefore more timely (and therefore more accurate, relative to current conditions) measurements. Such more timely measurements can lead to advantages in various uses and applications in which the measurements are used, including, for example, determination and display of data or feedback relating to treatment or the patient.

Additionally, using a sensing device for measurement of various ventilatory parameters, such as may include CO2, relative to sensing further from the patient's airway (e.g., at a ventilator or defibrillator) may provide other advantages. For example, it may allow for less tubing and gas flow between the patient's airway and the sensor, which may reduce inaccuracies associated with such additional tubing and gas flow, such as may include gas volume loss associated with tubing compliance and inaccuracies associated with lags related to diffusion through dead space, for example.

Still further, sensing, such as flow rate and pressure sensing, using a sensing device may be of use in tubing related problem detection (e.g., kinks, occlusion, or tangling) or detection of a leak in the breathing circuit. For example, this may include comparing the flow rate detected at the sensing device with the flow rate detected less proximately to the patient airway (e.g., at a ventilator, for example), where large differences between the two may indicate or suggest the presence of a leak or tubing related problem that is causing the less proximate sensing to not accurately reflect the patient airway flow rate.

Furthermore, as described in detail herein, in mechanical ventilation applications, such more timely measurements can reduce instances of, or reduce the magnitude of, patient ventilator asynchrony (PVA), which can cause serious risk or injury to patients. Reducing the magnitude of PVA may include or result from, for example, reducing the amount of time difference between initiation of a patient's inspiratory or expiratory effort and initiation of inspiration or expiration by a mechanical ventilator.

### [Leak Detection]

In some embodiments, a sensing device can be used in determining whether a gas leak exists, such as a leak in the gas flow conduit of a sensing device. For example, in some embodiments, one or more parameters, or a combination of parameters, determined using signaling from the sensing device, such as inspiratory or expiratory flow rates, and/or detected pressure inside of the gas flow conduit, may differ sharply from expected values. In some embodiments, a processor, using one or more algorithms, may use such determined flow rate and/or parameters in determining whether a leak is or may be present, and may, for example, present a message, alert or alarm to one or more care providers accordingly. Various features of embodiments of leak detection using a sensing device, as described herein, may incorporate or utilize aspects of features described in previously incorporated by reference U.S. Pat. No. 11,433,211, issued on Sep. 6, 2022 and entitled, “Flow Sensor for Ventilation.”

In some embodiments, measurement of the pressure outside of the conduit, which may reflect an ambient or environmental pressure, may be useful in various ways. For example, in instances in which ambient pressure is lower than sea level pressure, or substantially enough lower, such as may occur at altitude relative to sea level, measurement of the ambient pressure may allow for more accurate measurement of patient airway flow rates or pressures, such as by allowing correction or calibration or pressure and flow measurements to account for the lower ambient pressure at altitude. For example, mass flow sensors, such as thermal mass flow sensors, measure the mass of the gas molecules of the flow. However, it is often needed to determine and utilize volumetric flow, which is the volume of the flow. Volumetric flow can be computed from mass flow by factoring in the density of the gas. However, the density of a gas depends on conditions including the gas content (e.g., percentage O2 or other gases), the temperature and the pressure (e.g., atmospheric or ambient pressure). As such, to accurately compute volumetric flow based on mass flow sensor output, it may be necessary or desired to factor in the ambient pressure (and it may be necessary or desired to factor in the other conditions as well). While ambient (or atmospheric) pressure at sea level is often used, as altitude increases, ambient pressure decreases, such that the ambient pressure at altitude may vary significantly from the ambient pressure at sea level. Measurement of the pressure outside of the conduit may reflect ambient pressure (including at altitude), and therefore may be used in accurate, or corrected, measurement of volumetric flow based on mass flow sensor output, to factor in the actual ambient pressure (or correct for the effect of the pressure at altitude, relative to sea level pressure, on the measurement).

In some embodiments, a system is provided that includes a sensing device and at least one processor (whether at a sensing device, another device, or multiple devices), such as a processor of a medical or computing device or system, whether local, remote, or both, such as may include the use of one or more algorithms (although, in some embodiments a processor may be included in a sensing device, or also in a sensing device). The processor may receive signals from (or within) the sensing device, such as may include signals from the thermal mass flow sensor and pressure sensor(s) of the sensing device. Using these signals, the processor may determine measurements including flow rates and pressures, such as patient airway flow rates and pressures.

The processor may use such patient airway flow rate and pressure measurements in determining various parameters, states or conditions, such as parameters, states or conditions relating to treatment (e.g., ventilation or chest compression parameters) and/or parameters, states or conditions relating to the patient (e.g., physiological parameters or states, disease or disorder states).

Furthermore, processor may use the measurements or other determinations in determining and providing data or feedback, such as to one or more care providers. The determined or provided data or feedback may relate, for example, to provided treatment, or may be for care provider (or automatic) adjustment or optimization of parameters of provided treatment (e.g., parameters of provided BVM ventilation or chest compressions).

Various features of a processor and processing, and determination and providing of parameters and feedback, such as relating to treatment or the patient, and well as features of various computerized and medical devices, as described herein, may incorporate or utilize aspects of features described in previously incorporated by reference U.S. Pat. No. 11,433,211, issued on Sep. 6, 2022 and entitled, “Flow Sensor for Ventilation.”

In some embodiments, measured ventilatory parameters (where a ventilatory parameter may include any ventilation related parameter) may include or relate to, for example, one or more of: temperature, pressure (e.g., patient airway pressure, ambient pressure), flow rates (e.g., patient airway flow rate, mass flow rates, volumetric flow rates), concentrations of particular gases or compounds, air, CO2, end-tidal CO2, CO2 output (VCO2), oxygen (02), nitrogen (N), nitric oxide (NO) and others, whether such ventilatory parameters are measured based on sensing at or within a sensing device or elsewhere. In some embodiments, waveforms are determined, such as by one or more processors, that relate to or are determined based on one or measured, determined or calculated ventilatory parameter values over time. Furthermore, waveform morphologies associated with such waveforms may be analyzed and used in making various determinations, such as may be helpful or used in connection with ventilation or optimizing parameters, aspects or conditions associated with ventilation, such as may relate to the patient, treatment of the patient or other circumstances affecting the patient or patient care. This may include, for example, determining an associated type of ventilation or treatment(s) being provided (e.g., mechanical ventilation, BVM ventilation, CPR chest compressions), or determining a physiological or respiratory state of the patient (e.g., return of spontaneous circulation (ROSC), spontaneous breathing, agonal breathing, parameters relating to CPR chest compressions such as rate or depth of compressions), in breathing circuit leak detection, or in detecting or confirming correct endotracheal tube placement, for example. Additionally, in some embodiments, one or more CLC algorithms may be used in combination with determinations based on waveform morphology. For example, in an alternating chest compressions and mechanical ventilation protocol, a CLC algorithm may be used in, at detection of the end of a prescribed number of breaths, initiating mechanical chest compressions.

In some embodiments, flow rate and/or pressure measurements, such as patient airway flow and pressure measurements, which may include inspiratory and/or expiratory flow rate and pressure measurements (including flow rate and pressure measurements sensed at a sensing device and reflective of patient airway, inspiratory and expiratory flow rates and pressures), may be used in determining waveforms reflecting, e.g., patient airway flow rate or pressure over time. Additionally, in some embodiments, other waveforms may be generated, such as by one or more processors and based in part on factors including patient airway flow rate and/or pressure measurements over time, but also based in part on other factors or parameters, such as other ventilatory parameters. For example, such other waveforms may be based on waveform functions that mathematically define such waveforms based on mathematical combinations of various factors or parameters. For example, a volume waveform may be determine based on integration of flow, or a rate waveform may be determined based on a frequency of amplitude peaks, etc.

In some embodiments, one or more processors, executing one or more algorithms, and using data stored in one or more memories, may determine and generate such waveforms, for use in making various determinations and/or for display, such as to one or more care providers. In some embodiments, the data may include collected or historical patient airway flow rate and pressure measurement data over time, or collected or historical associated waveform data. In some embodiments, one or more algorithms, such as may include one or more machine learning models or algorithms, may be used in analyzing and making determinations based on such data, and may, for example, use collected or historical data as basis or training data.

In some embodiments, one or more processors may determine or analyze the morphology of such waveforms (where morphology can include any morphological aspects or features), such as to determine parameters, states or conditions associated with treatment or the patient, and may provide, present or display feedback or data based on such determinations. In some embodiments, the determined or provided data or feedback may relate, for example, to provided treatment, or may be for care provider (or automatic) adjustment or optimization of parameters of provided treatment (e.g., parameters of provided BVM ventilation or chest compressions).

In some embodiments, the processor may determine, or may be provided with as input, profiles of waveform morphology profiles associated with particular treatment or patient related parameters, states or conditions. In some embodiments, each of the morphological profiles may define a set or range of morphologies (such as may include values or ranges for one or more morphological features) of one or more waveforms that characterize one or more particular treatment or patient related parameters, states or conditions. The processor may analyze the morphology of a particular waveform, such as of a patient currently undergoing treatment, compare the waveform morphology to morphological profiles, and determine that the particular waveform morphology matches a morphological profile. Based on a match, the processor may determine that the particular waveform indicates the presence of, or the likely or possible presence of, one or more particular parameters, states or conditions characterized by the morphological profile(s). As such, the processor may use morphological profiles to match to particular waveforms, such as may relate to a patient undergoing treatment, to identify current patient or treatment conditions. In some embodiments, morphological profiles may also relate to degrees or magnitudes of a particular condition (e.g., a mild, moderate or severe form of a patient disease condition). Although embodiments are described in connection with morphological profiles, in other embodiments, analysis of waveform morphology and determination of associated conditions may be performed without use of profiles.

In some embodiments, morphological profiles may relate to treatment related conditions, such as, for example, that the patient is currently undergoing a specific type of treatment, such as BVM ventilation, mechanical ventilation, or chest compressions. Morphological profiles may also allow identification of particular parameters relating to a particular treatment. For example, as described in detail herein, even without chest compression sensor accelerometer data or other data such as compression pad impedance data (which may not be available), the processor may be able to identify not only that chest compressions have been or are being provided, but may also be able to identify portions of waveforms relating to particular chest compressions, as well as the timing or state of each chest compression (e.g. the positive phase when compression force is applied, etc.), and particular parameters such as the number of chest compressions applied in a current set and the compression rate. Analogously, with respect to BVM ventilation, the processor may be able to determine, e.g., the number of ventilations provided in the current set, the breath rate, and PIP, such as by analysis of a patient airway pressure waveform.

Additionally, in some embodiments, morphological profiles may relate to patient or patient physiology parameters, states or conditions. For example, morphological profiles may allow, or help in, the identification of the presence of (or lack of presence of), e.g., spontaneous breathing, ROSC or agonal breathing, as well as particular details thereof (e.g., spontaneous breathing rate).

Various morphological features may be used in various morphological profiles, or may otherwise be used in identifying various treatment or patient related parameters, states or conditions. For example, flow rate and pressure waveform morphological features (whether instantaneous or over a period of time) may include amplitude, leading edges, trailing edges, slope, rise time, fall time, hold time, plateaus, dips, peaks, notches, and others, as well as details associated with such features (e.g., magnitude or duration), or averages, thresholds or mathematical aspects of, e.g., repeated occurrences of such features, such as a magnitude of deviation, or comparisons between different features (e.g., ratio of rise time to fall time), for example.

Herein, in some instances, variations of a term may be utilized that may refer to the same or similar concepts, and certain terms may have meanings that are informed by a particular context. Various ventilation parameter related terms or abbreviations (e.g., PIP) may refer to ventilation related settings, even though the word “setting” may or may not be stated. Furthermore, reference to a ventilation parameter or parameter setting may be used to refer to the parameter in a conceptual or definitional sense, or the value associated with a particular setting (e.g., “PIP of 20 cm H2O”). In the ventilation field, a PEEP setting may sometimes be called a Baseline Airway Pressure (BAP) setting, with both terms referring to the same setting. As such, it should be noted that, herein, with reference to a setting, PEEP and BAP are to be understood to be used alternatively and to refer to the same setting. A user, as described herein, may include an individual operating, supervising or in whole or in part responsible for operation of a device such as a portable ventilator, even if, during a particular period of time while the device is operating, the user may not be interacting with the device.

An alert, alarm or feedback, as used herein, may be presented for the attention of a user, such as by being visually or audibly presented, such as via a display, graphical user interface (GUI) or speaker of a device. However, it may also include reference to conditions that are algorithmically identified, recognized or determined by a computerized device and not necessarily presented or displayed. Herein, the term optimizing may include, for example, improvement or improved operation in one or more aspects, for example, relative to an actual, potential or hypothetical less optimized situation or less optimized operation.

Herein, a ventilator can include, for example, a ventilator or ventilation device, apparatus or system, whether or not portable, and whether or not functionality other than ventilation aspects is provided by the device, apparatus or system, including manual ventilation, bag ventilation, ventilation using an endotracheal tube and mechanical ventilation. Herein, the term adjusting can include changing as well as not changing or maintaining without change, as may be appropriate. Herein, a determined parameter value can include a determined estimated or determined approximated value for the parameter. Herein, the term monitoring can refer to or include, for example, monitoring or tracking performed by a computerized device utilizing one or more algorithms and not by a person or user, or monitoring by a person or user, or both. Herein, the term continuous can include, among other things, on a periodic basis (with identical or different periods), on a frequent basis, on a repeated basis, or cyclically, for example.

In some embodiments, pressure and/or flow sensing using a sensing device may be used in closed loop control of one or more ventilation parameters or other treatment related parameters, such as CPR chest compression related parameters. The term closed loop control, as used herein, may refer to control of one or more treatment related or patient related parameters, such as with relatively little or no required user action, participation or intervention, and can include reference to, but is not limited to reference to, fully automated or fully automatically regulated control. Closed loop control may include, for example, device facilitated or algorithmically facilitated tracking, control and adjustment of one or more parameters, which may or may not include user involvement or participation. Where user involvement or participation is included, it may include, for example, confirming a suggested or recommended ventilation setting change or configuration, deciding on implementing a course of action, selecting one of several suggested courses of action, responding to a presented alert or alarm, or other decisions, choices or actions. User involvement or participation could also include, for example, setting or changing a parameter, where a closed loop control algorithm proceeds from there, initially according to the user-set or user-changed parameter setting. In various embodiments, if there is user involvement, it may be, for example, among other things, in whole or in part user-initiated, or in whole or in part prompted, suggested, recommended or required.

In some embodiments, closed loop control may be utilized but may be subject to manual adjustment or override by the user. For example, in some embodiments, although FiO2 and PEEP (BAP) may be algorithmically and automatically controlled, a user may be able to intervene and manually change the FiO2 and/or PEEP (BAP) setting. In some embodiments, following any manual adjustments, closed loop control of FiO2 and/or PEEP (BAP) may resume from that point, at least until any further manual adjustments are made.

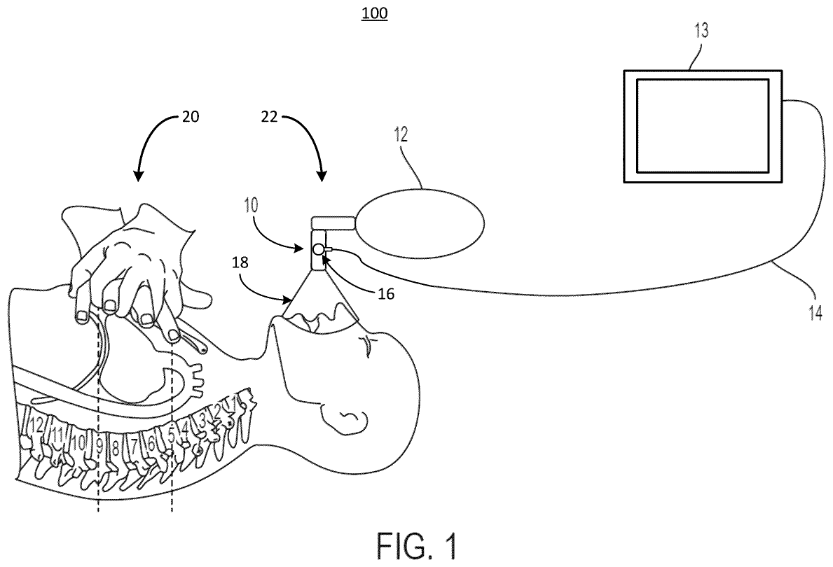

FIG. 1 illustrates an example emergency care environment 100 including BVM ventilation with use of a sensing device 10, in accordance with some embodiments. The sensing device 10 is coupled between a bag 12 and a mask 18 of a BVM system 22, with the mask 18 being on the patient and coupled with the patient's airway. The BVM system 22 may be operated by a care provider to deliver breathing gas, such as air or oxygen enriched gas, to the patient. Additionally, in some embodiments, chest compressions 20 may be delivered to the patient, such as by the same care provider providing the manual ventilation or another care provider, such as intermittently with breaths delivered using the BVM system (e.g., in an example 30:2 protocol, where 30 chest compressions may be provided, then 2 ventilation breaths, repeatedly), or by a mechanical device such as a chest compressor, such as a band based or piston based chest compressor. The sensing device 10 includes a thermal flow sensor, such as a hot wire anemometer, for measurement of gas flow inside of a gas flow conduit of the sensing device 10, such as an inspiratory flow or expiratory flow. The sensing device 10 includes one or more pressure sensors for measurement of the pressure inside of the conduit. The sensing device 10 may include an absolute pressure sensor for measurement of the pressure outside of the conduit. The sensing may also include one or more flow conditioners for conditioning the flow of gas inside of the conduit, such for improving the accuracy of inspiratory pressure or expiratory pressure measurements. The sensing device 10 may further include an integrated CO2 sensor.

Additionally, the sensing device 10 may be used with or may include a rotatable connection adapter 16 for cable 14 connection to another device 13, such as a medical device (e.g., defibrillator), other medical device, patient monitor, portable computing device (e.g., a tablet of a care provider), although, in other embodiments, wireless connection may be used or may also be used. In the embodiment depicted in FIG. 1, the sensing device 10 is shown in use with BVM ventilation. However, in some embodiments, a sensing device 10 may be used with mechanical ventilation, in which case the sensing device 10 may be connected or coupled, such as by wire or wirelessly, to a mechanical ventilator or other device, such as via a rotatable connection adapter.

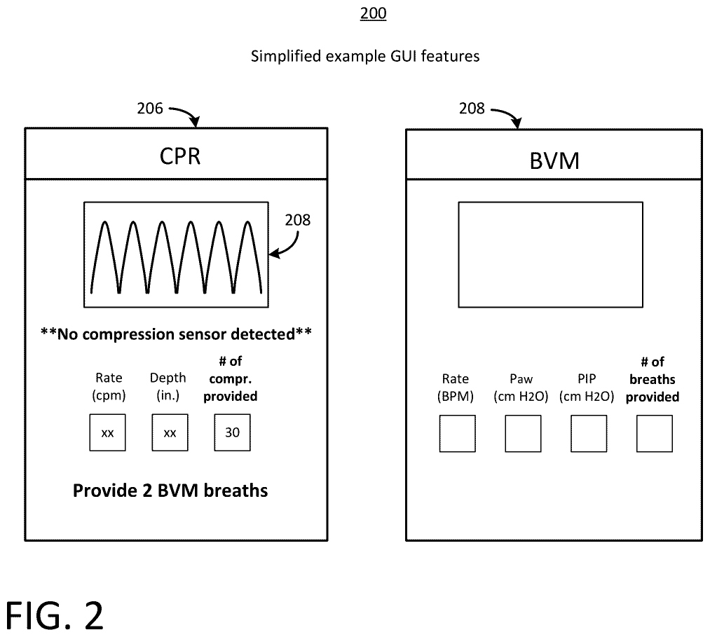

FIG. 2. Illustrates portions 206, 208 of a simplified display, or display/graphical user interface (GUI), including “BVM” (which, in display 208 FIG. 2, refers to BVM ventilation treatment) and “CPR” (which, in display 206 of FIG. 2, refers to CPR chest compression treatment) parameters and/or feedback determined using measurements for which a sensing device was used. The display may be, for example, a display of a medical device, monitor, or portable computing device. In some embodiments, signals from a sensing device may be used, such as by one or more processors, in flow rate and pressure measurements that are used in determining or measuring various parameters, such flow, volume and pressure parameters, which may be used in determining displayed parameters and feedback. Although both BVM ventilation and CPR chest compression feedback are shown, in some embodiments, only BVM ventilation or CPR chest compression feedback may be provided, such as when only BVM ventilation or CPR chest compressions are provided to the patient, and/or other feedback may be provided for one or more other treatments. In some embodiments, the display may also include displayed waveforms, such as patient airway flow rate or pressure waveforms. Various features of ventilation and chest compression parameter and/or feedback determination and presentation, as described herein, may incorporate or utilize aspects of features described in previously incorporated by reference U.S. Pat. No. 11,433,211, issued on Sep. 6, 2022 and entitled, “Flow Sensor for Ventilation.”

In some embodiments, during the providing of alternating ventilation breaths and chest compressions, such as according to a 30:2 protocol, parameters and/or feedback may be provided relating to each or both. For example, while chest compressions are being provided, chest compression parameters and/or feedback may be displayed, and when ventilation is being provided, ventilation parameters and/or feedback may be displayed. Additionally, feedback may be provided that is associated with both, such as feedback including an indication or alert to stop provide ventilation breaths or provide chest compressions, or a message, alert or alarm that both BVM ventilation and chest compressions are being provided concurrently, against protocol, as described further herein.

In some embodiments, during ventilation treatment, flow and internal pressure sensing of the device may be used in determining and presenting ventilation parameters and/or feedback, such as an indication of provided breath rate, patient airway pressure, peak pressure, peak inspiratory pressure (PIP) and/or number of breaths provided (such as during the providing of a set of 2 BVM breaths during a 30:2 protocol).

In various embodiments, a sensing device may be used in treatments including chest compressions with or without use of a chest compression sensor (or “puck”) or other sensing arrangements (e.g., use of compression pad impedance data). In some situations and examples, during the providing of chest compressions, one or more accelerometers, such as may be incorporated into a chest compression sensor applied to the chest of the patient, may be used in determining chest compression parameters and/or feedback, such as may include or relate to rate, depth, force, release, and angle of chest compressions. In situations in which accelerometer sensing is used in determination of chest compression parameters and/or feedback, flow and/or pressure sensing of a sensing device may be used, for example, in confirming or enhancing the reliability or accuracy of certain measurements, including chest compression rate and depth.

In some examples, however, a chest compression sensor may not be used (e.g., in an emergency situation when it is not available), or may not function or may malfunction, such that accurate accelerometer sensing may be unavailable. In such situations, for example, flow and/or pressure sensing of a sensing device may be used in providing chest compression feedback, including, e.g., rate. In some embodiments, for example, as described herein, a pressure waveform, which may represent pressure as it changes over time, may be used in determining (or confirming or enhancing) chest compression parameter and/or feedback measurements, such as chest compression rate and/or depth. In various embodiments, a sensing device may be used with various forms of chest compressions, including manual and automated chest compressions (e.g., band or piston based).

For example, the depicted simplified displays 206, 208 may be provided during the providing of BVM ventilation and chest compressions according to a 30:2 protocol. In some embodiments, the displays 206 may include one or more waveforms 208, such as patient airway flow rate or pressure over time waveforms (as shown, waveform 208 represents a simplified example of a patient airway pressure over time waveform during the providing of a set of chest compressions). In the example depicted, the system has determined (or it has been input into the system, such as by a care provider) that no chest compression sensor is detected, such as because it is unavailable, not functioning or malfunctioning. However, flow and/or pressure sensing of a sensing device may be used in providing chest compression feedback, such as may include compression rate (e.g., in compressions per minute (CPM)) and depth (e.g., in inches), as well as total number of compression provided (e.g., in a set of 30). In the example depicted, in the chest compression related display 206, “30” is indicated as the “# of compressions,” indicating that the care provider has just completed a set of 30 compressions (as may, in some embodiments, be determined or confirmed using, e.g., pressure sensing of a sensing device). At this point, the display 206 may also include feedback such as an alert instructing the providing of BVM breaths (instead of chest compressions), in accordance with the 30:2 protocol. Although not shown in display 208, since, in the example, BVM breaths are not currently being provided, when BVM breaths are provided, the BVM ventilation related display 208 may show BVM ventilation related parameters and/or feedback, such as breath rate, patient airway pressure, PIP and “# of breaths provided”, and these displayed values may change as the parameter values change during treatment. Additionally, in accordance with the 30:2 protocol, when a set of 2 breaths are completed, a message or alert may be provided to instruct to provide chest compressions (instead of BVM ventilation breaths).

Additionally, in some embodiments, if a chest compression sensor is determined to be present and being used, and chest compressions are detected from, e.g., patient airway flow rate waveform morphology (as described herein), and yet a sensing arrangement of the compression sensor (e.g., accelerometer or compression pad impedance) indicates that no chest compressions are being applied, this may signal, and be used in determining and providing feedback relating to, a problem with the compressor that needs to be addressed, such as a need to re-apply, re-accommodate or reposition it. Furthermore, in some embodiments, if a sensing arrangement of the compression sensor indicates that chest compressions are being applied, and yet analysis of patient airway flow rate morphology appears to indicate that no chest compressions are being applied, this may signal, and be used in providing feedback relating to, a situation in which the patient's lung(s) may be collapsed, and, for example, BAP or additional BAP may be needed to reopen them, for example.

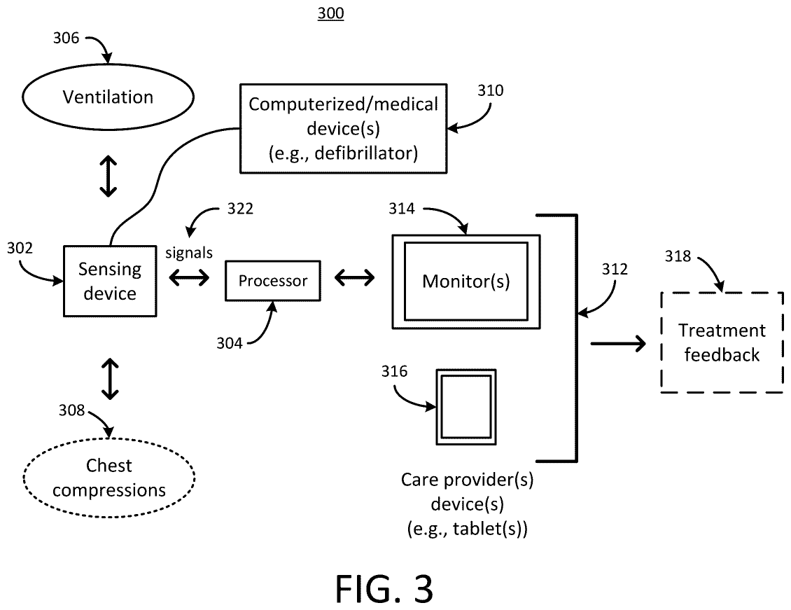

FIG. 3 is a block diagram 300 illustrating an example of use of a sensing device 302 in ventilation treatment 306 and potentially also chest compression treatment 308, and/or other treatments, such as defibrillation or pacing. The sensing device 302 may include features such as a thermal flow sensor, internal and external pressure sensing, one or more flow conditioners, an integrated CO2 sensor, and a rotatable connection adapter. The sensing device 302 may be coupled, such as by cable, to one or more other devices, such as computerized or medical device 310 (e.g., a ventilator, defibrillator, portable computing device or other device), and may additionally be coupled, such as by wireless coupling to one or more other devices or systems.

The sensing device 302 may be coupled to one or more processors 304. For example, the processor(s) 304 may be part of a medical or computing device, such as a device to which the sensing device 302 is coupled, or part of another device or system that receives signals, such as flow and pressure sensing signals from the sensing device 302, whether directly or indirectly (such as via one or more intermediary devices or systems). However, in some embodiments, the processor(s) 304 may be included in whole or in part within the sensing device 302. The processor(s) 304, such as along with memory, using the signals 322 as input, may use one or algorithms in, e.g., determining flow rate, volume and pressure measurements using the signals, and may use the determined measurements in determining various parameters and/or feedback, such as treatment feedback 318 (e.g., ventilation or chest compression feedback). The feedback 318 (e.g., visual, audio and/or haptic feedback) may be presented on or using one or more devices 312, such as one or more monitors 314 (e.g., patient monitors or monitors of medical devices) and/or one or more devices 316 of one or more care providers (e.g., one or more computing devices, portable computing devices, tablets or smart phones).

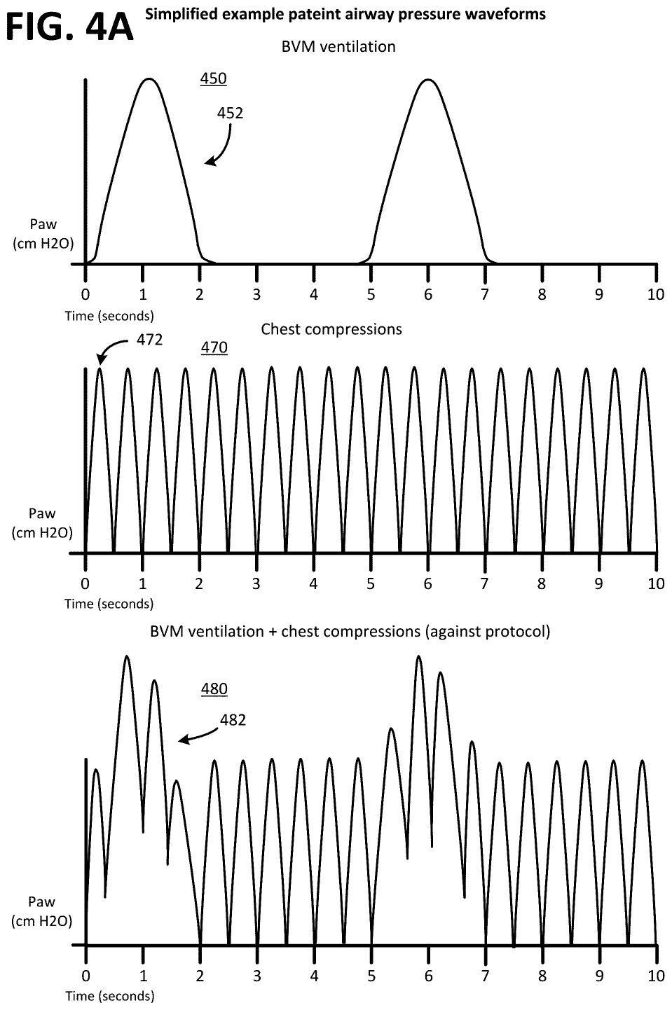

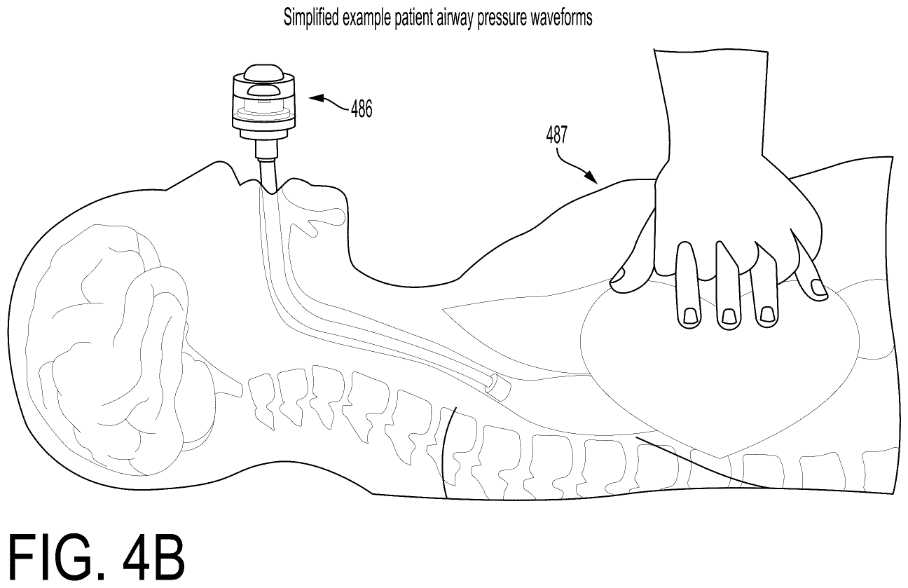

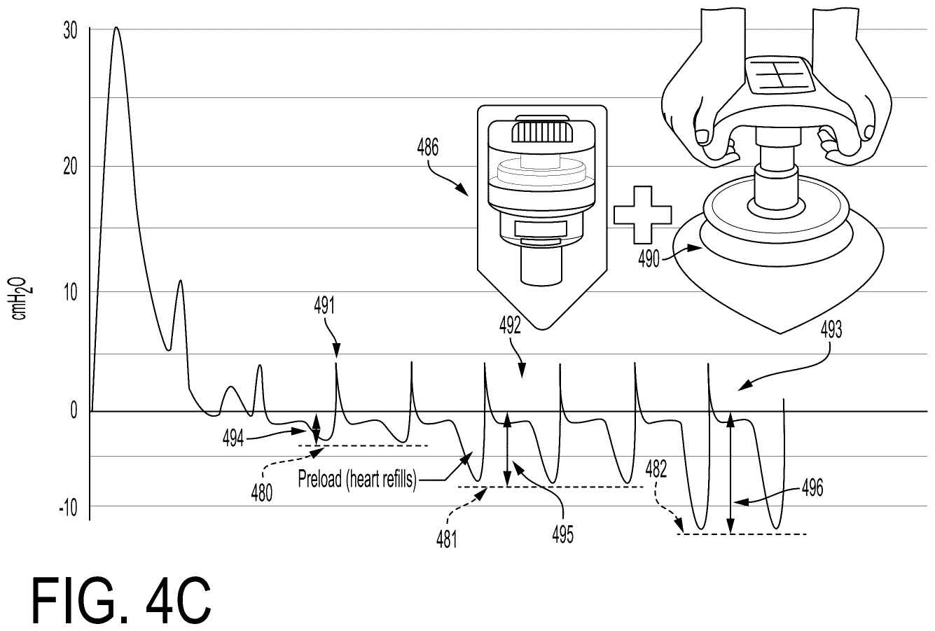

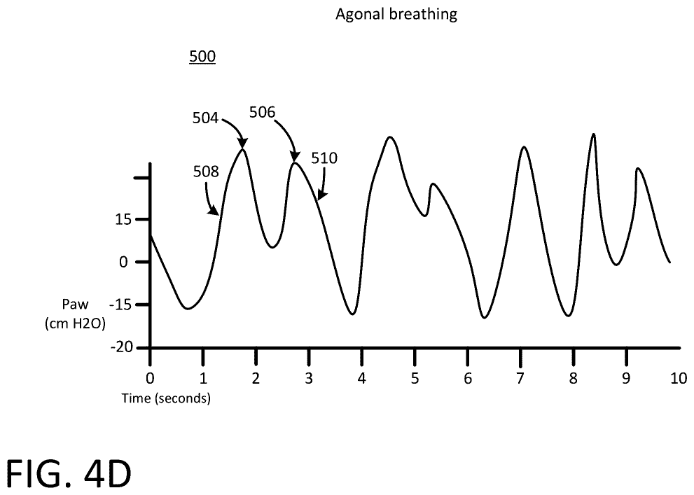

FIG. 4A illustrates simplified example patient airway pressure waveforms 450, 470, 480 associated with the providing of BVM ventilation, chest compressions, and both BVM ventilations and chest compressions (against protocol). It is noted that the waveforms 450 are simplified in order to clearly illustrate particular features, and are not intended to exactly represent actual patient airway pressure waveforms.

Simplified waveform 450 illustrates a waveform that may occur during the providing of BVM ventilation. Each of the two depicted raised portions 452, occurring roughly between 0-2 seconds and 5-7 seconds, represents a pressure increase followed by a pressure decrease corresponding with, respectively, the squeezing of a BVM bag to provide the inspiratory period of a breath, causing an increase in patient airway pressure, followed by the release of the bag causing an expiratory period, causing a decrease in patient airway pressure. In some embodiments, the morphology of the waveform 450, including the raised portions, the timing of the raised portions and portions thereof, and the periods of low or zero pressure between them, may be used, for example, in detecting individual provided breaths and/or portions thereof, as well as associated parameters (e.g., rate, patient airway pressure, PIP, number of breaths provided). In some embodiments, morphological profiles, as previously described, may be used in this regard (e.g., the waveform 450 may be matched to a morphological profile associated with BVM ventilations, in order to determine that BVM ventilation is being provided, or to determine particular parameters associated with the provided BVM ventilation).

Simplified waveform 470 illustrates a waveform that may occur during the providing of chest compressions. Each of the depicted twenty raised portions 472, occurring roughly every one half second, represents a pressure increase followed by a pressure decrease corresponding with, respectively, a compression phase (during which force is applied to compress the chest), followed by a release phase (during which no force is applied and the chest expands). In some embodiments, the morphology of the waveform 470, including the raised portions, the timing of the raised portions and portions thereof, and the periods of low or zero pressure between them, may be used, for example, in detecting or confirming detection of chest compression rate and/or depth, among other potential parameters. In some embodiments, morphological profiles, as previously described, may be used in this regard (e.g., a morphological profile associated with the providing of chest compressions).

Simplified waveform 480 illustrates a waveform that may occur during the providing of BVM ventilation concurrently with chest compressions as depicted in waveforms 450 and 470. This is contrary to the 30:2 treatment protocol, may occur by accident, and may cause patient injury, such as by causing a high patient airway pressure cause lung injury, as well as leading to ineffective treatment. The two overall raised portions 482, occurring roughly between 0-2 seconds and 5-7 seconds, correspond to periods during which BVM breaths and chest compressions are provided simultaneously, leading to high patient airway pressures. This waveform morphology may be used to determine that it indicates that BVM ventilation breaths and chest compressions are, or may be, being provided concurrently, against protocol. In some embodiments, morphological profiles, as previously described, may be used in this regard (e.g., a morphological profile associated with the providing of BVM ventilation treatment concurrently with chest compressions). As such, in some embodiments, the processor may further be used to determine and provide feedback accordingly, such as a message, alert or alarm to alert the care provider(s) of this, and to instruct them to discontinue the erroneous procedure, for example.

FIG. 4B is a simplified illustration of the providing of chest compressions 487 including use of an impedance threshold device (ITD) 486. FIG. 4C illustrates simplified example patient airway pressure waveforms associated with the providing of conventional chest compressions, chest compressions including use of an ITD, and chest compressions with use of an ITD and an active compression decompression (ACD) device.

During conventional manually applied chest compressions (without the use of an ITD or an ACD device), during a positive phase of a compression cycle, when pressure is applied, positive pressure generated during a compression may help circulate blood forward to peripheral tissues of the body. During a negative phase, the natural recoil of the chest may generate a vacuum, or negative pressure, that helps draw blood back to refill, or more quickly or completely refill, the heart. This may be called preload of the heart, and may be associated with effective CPR chest compressions. An ACD device and/or an ITD may be used, separately or together, to increase negative pressure during the decompression phase of a compression cycle, which may increase or speed preload and lead to more effective CPR chest compressions.

CPR chest compressions with an ACD device may be performed, for example, using a handheld device including a suction cup that adheres to the chest of a patient, which can be pulled up by the user. During the negative or decompression phase, instead of relying only on the natural recoil of the chest, the ACD device actively lifts the chest, enhancing chest wall expansion, increasing vacuum and negative pressure, thereby enhancing preload of the heart, and may thereby enhance the effectiveness of CPR chest compressions.

An ITD, such as ITD 486, may be a non-invasive device that can be used in providing intrathoracic pressure regulation (IPR) therapy during the providing of CPR chest compressions. An ITD may couple with a patient's airway, such as via a patient circuit or attachment to a face mask. The ITD includes a valve that allows air to be expelled during a positive phase of a chest compression cycle, but can limit or prevent the flow of air inward during the negative phase, thus increasing negative pressure and enhancing preload of the heart. In some implementations, both an ACD device and an ITD may be used, which may provide more negative pressure than use of either separately.

In FIG. 4C, for illustration purposes, simplified patient airway pressure waveforms corresponding with three sets 491, 492, 493 of two compression cycles each are depicted, corresponding with, respectively, the providing of conventional chest compressions, the providing of chest compressions including us of an ITD 486, and the providing of chest compressions including use of an ITD 486 and an ACD device 490.

In some embodiments, a patient airway pressure waveform may be generated using a sensing device. The waveform may include a negative pressure dip associated with use of an ACD device, an ITD, or both. In some embodiments, one or more morphological features associated with the negative pressure dip may be used to determine if either or both devices are being used, or to determine if either or both devices are being used effectively or properly, so as to produce an expected negative pressure dip. For example, morphological features associated with the dip, such as maximum depth/negative pressure or slope and/or duration, or changes in pressure, may be associated with use of one or both devices, or effective use of one or both devices.

For example, In FIG. 4C, the set 491 of chest compression cycles associated with the providing of conventional chest compressions, without use of an ITD or an ACD device, shows an example negative pressure dip 494 of approximately −3 cm H2O, as indicated by broken line 480 (or, in other examples, this dip may be e.g., between −1 to −5 cm H2O). Chest compressions provided with us of an ITD may include a much larger negative pressure dip, due to the effect of the ITD as described herein. As such, the set 492 of chest compression cycles associated with the providing of chest compressions with use of an ITD 486 shows an example negative pressure dip 495 of approximately −7 cm H2O, as indicated by broken line 481 (or, e.g., between −5 and −9 cm H2O). While not depicted, chest compressions including use of an ACD device, without use of an ITD, may include a negative pressure dip that is also greater than that of the providing of conventional chest compressions, but less than that of the providing of chest compressions with an ITD and an ACD device, for example. Finally, the set 493 of chest compressions cycles associated with the providing of chest compressions including use of an ITD 486 and an ACD device 490, which may include a negative dip that is greater than that of the providing of conventional chest compressions or chest compressions with either an ITD or an ACD device alone, such as, e.g., −12 cm H2O (or, e.g., between −10 cm H2O and −14 cm H2O). As such, in some embodiments, patient airway pressure negative dips, for example, of up to a particular threshold (e.g., as shown by the broken lines 480, 481, 482) may provide an indication or suggestion confirming that chest compressions are occurring without use of an ITD or ACD device, whereas dips reaching a certain threshold or range may provide an indication or suggestion confirming that chest compressions are occurring with use of an ITD, or with an ACD, or with both, or mechanical chest compressions without an ACD device or ITD, as described herein.

In the examples depicted in FIG. 4C, maximum negative pressure dips are an example of a pressure waveform morphological characteristic that may be used to suggest or detect the type of treatment being provided, or to suggest or detect whether the type of treatment being provided is being provided correctly or being sensed or measured correctly, or whether the system is working properly (e.g., not leaking, etc). For example, if a type of treatment is being applied incorrectly or measured incorrectly (e.g., including inaccurate pressure measurements), then unexpected pressure dips may suggest or indicate this, which may in turn be used to trigger a prompt for system of care provider checks, for example. Conversely, dips in expected ranges may be taken as some indication that treatment is being provided correctly and measured accurately (e.g., check the device(s), system, patient circuit, or the procedures being used). When treatment is being provided correctly (e.g., negative pressure measured indicates the presence of an ITD properly in use), then there may be an indication as such, e.g., light or display on a user interface or feedback device confirming that use of the ITD results in the desired ranges of negative pressure.

Additionally, while examples are providing with regard to a patient airway pressure waveform, in some embodiments, patient airway flow waveforms, or other waveforms (e.g., flow rate or other ventilatory parameter waveforms such as may be, for example, based on signaling from a sensing device, or otherwise), may also be utilized, in combination with appropriate associated morphological characteristics (e.g., appropriate associated flow rates, flow rates reaching particular thresholds, flow rates within particular ranges, etc.). For example, patient airway flow rate waveforms and their morphologies may vary with treatment types (e.g., conventional chest compressions, chest compressions with an ITD or an ACD device, or chest compressions with both), for example, in some ways analogously to that of patient airway pressure. For example, chest compressions including use of an ITD or ACD device may include greater expiratory flow rates (or flow rates beyond certain thresholds or within in certain greater ranges, or greater slope, etc.) than conventional chest compressions, and chest compressions including use of both an ITD and an ACD device may have still greater expiratory flow rates. Furthermore, mechanical chest compressions (without use of an ITD or an ACD device) may include more precisely matching flow rate waveforms associated with individual compression cycles, for example. Still further, pressure and flow rate waveforms associated with mechanical ACD chest compressions may differ from those manual ACD compressions (e.g., with a care provider using an ACD device in performing chest compressions). For example, while both may include a greater negative dip (e.g., in pressure or flow rate) during decompression phases relative to conventional chest compressions, automated ACD chest compressions may include additional features associated with the mechanical aspects. These may include, for example, more precisely matching waveform portions associated with individual compression cycles, and sharper, more regular, or more defined slopes or level portions, for example.

Furthermore, while maximum negative pressure is used as an example of a morphological characteristic that may be associated with particular treatments, various other morphological characteristics, of pressure, flow, other ventilatory parameter waveforms, or other waveforms, or combinations or sequences of such characteristics, including, e.g., instantaneous characteristics or characteristics over time, may be utilized. For example, for a pressure waveform, utilized morphological characteristics could include, e.g., maximum negative pressure, maximum negative pressure being above a particular threshold or within a particular range, maximum average negative pressure over time, rise and fall portions, leading and trailing edges, or others (or combinations or sequences thereof, or combinations between different waveforms or over multiple compressions cycles, etc.).

For example, mechanical chest compressions, but not including use of an ACD device (e.g., belt or plunger based mechanical chest compressions) may be associated with particular patient airway pressure or flow (or other) waveform characteristics. For example, such mechanical chest compressions may include a steep initial slope, due to rapid action during the positive phase, and/or a flatter peak, due to a long or pronounced time between cycles. Additionally, an identifying morphological characteristic of such mechanical compressions could include more precisely matching waveforms associated with individual compressions, due to the very consistent action of the mechanical system for each compression cycle. This is in contrast to the airway pressure or flow waveform characteristics associated with manual compressions, which may be, for example, more sinusoidal in nature with less consistency in the shape of the waveform in comparison to that which arises from automated mechanical CPR devices.

Furthermore, in some embodiments, one or more algorithms, such as may include one or more machine learning models, may be used in, e.g., identifying detecting waveform morphological characteristics associated with particular treatments, correct or accurate measurements associated with such treatments, etc. For example, historical data associated with waveform characteristics corresponding with instances of application of a particular treatment may be saved in a database, and one or more algorithms or machine learning models could use such data, e.g., as training data, to refine definition of morphological characteristics or patterns associated with particular treatments, to better identify such treatments, or correct or incorrect application or measurement of such treatments, etc.

FIG. 4D illustrates a simplified example patient airway pressure waveform 500 associated with agonal breathing (which may include, e.g., gasping for air), such may occur during periods of patient distress, and which may result in ineffective oxygenation from breathing. The waveform 500 may include a disorganized or chaotic morphology including pressure peaks 504, 506 of various different amplitudes, rise and fall portions with various different slopes 508, 510, leading and trailing edges, and other drastically varying characteristics, which may be indicative of agonal breathing. In some embodiments, a processor, using one or more algorithms, may recognize this waveform morphology, determine accordingly that agonal breathing is or may be present, and alert and/or instruct the care provider to take appropriate measures, for example. In some embodiments, morphological profiles, as previously described, may be used in this regard (e.g., a morphological profile associated with agonal breathing).

In some embodiments, patient airway flow and/or pressure waveform morphologies, potentially along with other parameters including end CO2 or a CO2 waveform over time, may be used in detecting ROSC. For example, ROSC may be accompanied by greater patient airway pressure variations, as a result of the spontaneous breathing, or potentially gasping or uneven breathing that may occur at the start of spontaneous breathing. As such, a patient airway pressure waveform during a period leading up to, and then including, ROSC, may result in particular waveform morphologies. In some embodiments, a processor, using one or more algorithms, may be used to analyze the waveform, recognize the morphology, and determine that ROSC has occurred or may be occurring, and a message, alert or instruction may be accordingly be provided to one or more care providers. In some embodiments, morphological profiles, as previously described, may be used in this regard (e.g., a morphological profile associated with ROSC). For example, in some embodiments, a CO2 waveform may be used along with a patient airway volumetric flow rate waveform in computing volumetric CO2 (e.g., total volume of CO2 expired per minute). This may be used to provide indicators of global metabolic, circulatory and respiration quality, which in turn may be used in detecting ROSC.

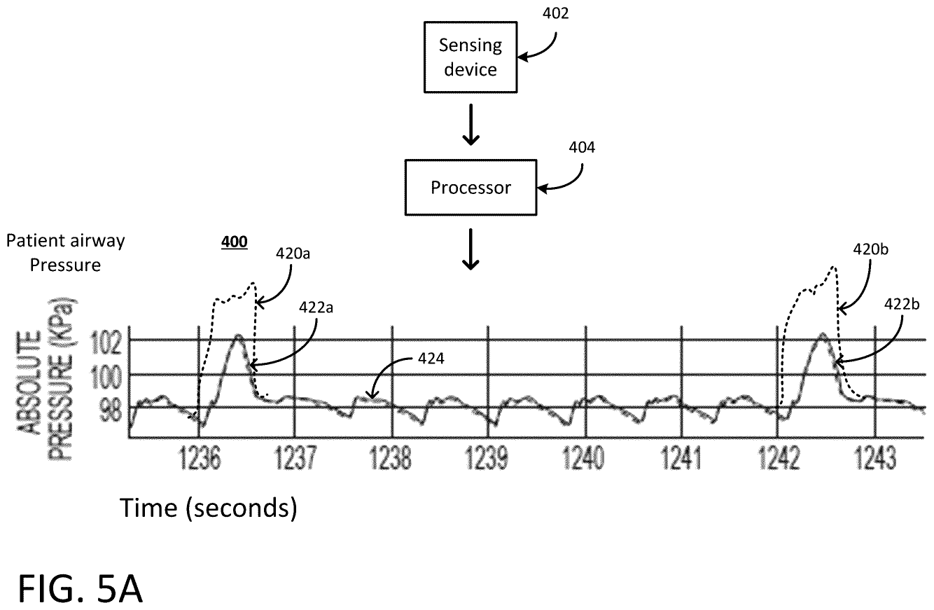

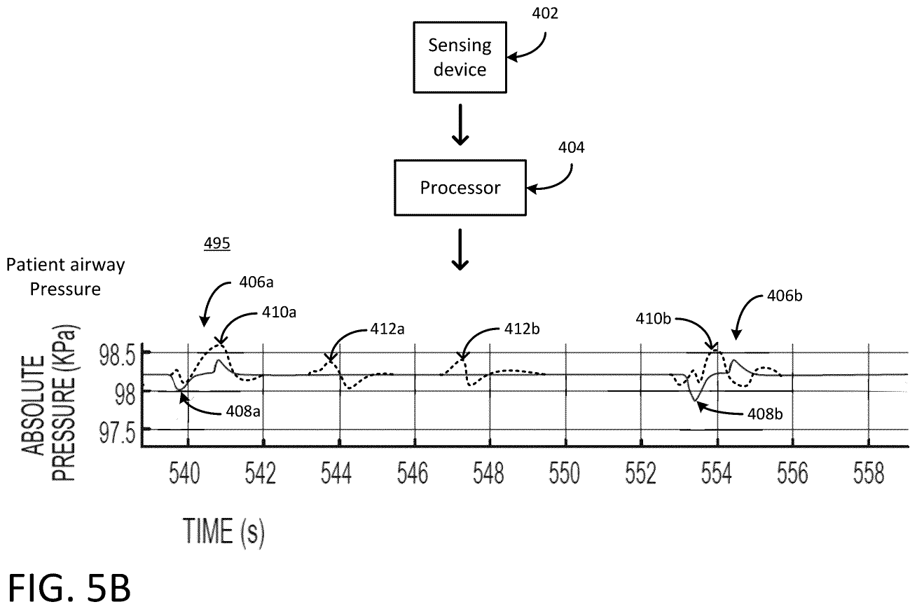

FIG. 5A illustrates an example patient airway pressure waveform 550 associated with BVM ventilation alternated with chest compression treatment, which can be generated and used based on use of a sensing device, such as may be associated with internal pressure sensing of a sensing device, reflective of patient airway pressure. As conceptually depicted in FIG. 4, the waveform 400 may be determined based on signals received by a processor 404 from a sensing device 402 during treatment of a patient.

As depicted, the waveform 400 results from absolute pressure sensing inside of a gas flow conduit of the sensing device 402 (however, in other embodiments, as described herein, differential pressure sensing inside of the gas flow conduit of the sensing device 402, along with absolute pressure sensing outside of the conduit, but be used to determine the pressure inside of the conduit).

The waveform 400 may result from alternating BVM ventilation breaths and chest compressions (e.g., in a protocol such as a 30:2 protocol). The raised portions 422a,b, occurring at approximately between 1236-1236.75 seconds and between 1242-1242.75 seconds, each generally correspond with a provided BVM breath, during which no chest compressions are provided. The smaller raised portions 424 each generally correspond with a provided chest compression.

A variation of the waveform 400 is shown by the dotted features 420a,b, which, in the variation, replace the solid line features during those periods. This variation may occur in a situation in which chest compressions, against protocol and potentially dangerously, continue even during periods of BVM ventilation breaths, causing pressure spikes during those periods. In some embodiments, the processor 404, using one or more algorithms, recognizes this waveform morphology as being associated with concurrent providing of BVM ventilation breaths and chest compressions. In some embodiments, morphological profiles, as previously described, may be used in this regard (e.g., a morphological profile associated with the providing of BVM ventilation treatment concurrently with chest compressions). The processor may accordingly generate and present a message, alarm, instructions or other feedback to one or more care providers to alert the care provider of this dangerous error, for immediate correction.

The spontaneous breathing pressure waveform 495 of FIG. 5B exhibits a morphology including a generally regular, repeating pattern for each breath, with approximately constant pressure between breaths. This morphology may be associated with normal breathing. Each of the periods 406a,b, from approximately between seconds 539-541 and between seconds 553-555, are associated with a spontaneous patient breath, starting with a lower pressure resulting from pressure generated by downward movement of the patient's diaphragm, followed by higher pressure resulting from expiration, with periods of approximately constant pressure between spontaneous breaths. However, some noticeable differences between the periods associated with each breath are present; in particular, the low pressure dip 408a in the first pattern is noticeably lower than the low pressure dip 408b in the second pattern. Spontaneous breaths are never identical, so some differences, such as this, may be present in normal breathing.

However, spontaneous breathing patient airway flow and/or pressure waveform morphologies, if they exhibit certain features or combinations of features (or if they exceed some specified threshold associated with some features and their magnitudes, for example, where such threshold may be determined algorithmically and/or using one or more machine learning models), may be indicative of a patient who is experiencing agonal respiration. Agonal respiration is an abnormal pattern of breathing that may include gasping or labored breathing. Given the strained and chaotic nature of agonal breathing, it may result in patient airway flow and pressure waveform morphologies that exhibit associated features, such as abnormal or chaotic pattern, steep slopes or high or low amplitude spikes or dips. For example, a variation of the waveform 500 is shown by dotted features 410a,b and 412a,b, which, in the variation, replace the solid line features during those periods. The variation includes clearly morphologically unmatching and irregular breath patterns 410a,b as well as clearly morphologically unmatching and irregular periods 412a,b within the otherwise relatively constant pressure period between breath patterns. As such, the variation may exhibit features sufficient to cause the processor 404 to identify it as being, or likely being, associated with agonal breathing.

As such, in some embodiments, the processor 404, using one or more algorithms (and potentially using one or more morphological profiles) may detect such characteristics and determine that agonal breathing is or may be present (and may detect normal breathing morphology as well, when normal breathing is present). For example, in agonal breathing, the patterns associated with breaths may be irregular and different from each other (e.g., different pressure highs and lows, different slopes, different amounts of time). Additionally, the periods between breaths may be irregular and different from each other, and may include irregular pressure spikes or dips. The processor 404 may detect agonal breathing, or likely agonal breathing, from such detected morphologies, and may determine and present messaging or feedback to alert one or more care providers of the condition and instruct accordingly.

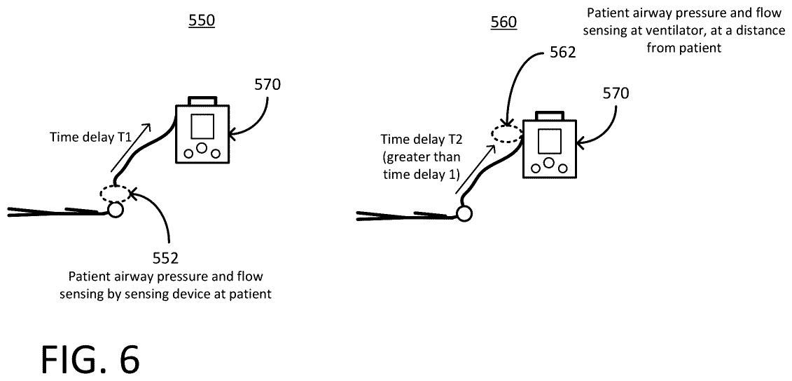

FIG. 6 illustrates a comparison, in mechanical ventilation, of patient airway pressure sensing 552 using a sensing device, according to some embodiments, relative to patient airway pressure sensing 562 at a different connected device, such as a ventilator 570. As described previously, in some embodiments, use of a sensing device for sensing in measurement of patient airway flow rates and pressures may provide advantages, for example, relative to flow and pressure measurements made using sensing occurring at a different device that is further from the patient's airway than the sensing device, where such different device may be connected by wire or tubing to the patient (e.g., a ventilator, defibrillator, other medical device or other computing device). Particularly, since the sensing device senses patient airway flow rate and pressure extremely close to the patient's airway, as opposed to sensing at another connected device that is not as close to the patient's airway, use of the sensing device may allow for less delayed or non-delayed measurements, and thus measurements that more accurately reflect current conditions, relative to measurements that use sensing farther from the patient's airway. This, in turn, can lead to various advantages, such as in determination and providing of data based on the measurements, and treatment or feedback based on the measurements.

In some embodiments, such as in the embodiment depicted in FIG. 6, a sensing device is used with mechanical ventilation and coupled with a mechanical ventilator 570, such as a portable or field ventilator (or, e.g., a defibrillator, such as may be in communicative contact with a ventilator). In example environment 550, patient airway pressure sensing 552 takes place using a sensing device coupled with, and located very close to, the patient airway. The associated pressure signal, conveyed electrically via a cable, takes a very small amount of time to reach the ventilator 570 (or defibrillator or other device(s)), causing only a very small delay in what the processor of the ventilator treats as the current patient airway pressure, including in determining when to initiate inspiration and expiration periods, relative to the actual current patient airway pressure, which time delay is represented as T1 (a latency). In example 560, pressure sensing takes place at the ventilator 570 (or defibrillator or other device(s)) itself, connected to the patient via tubing. The associated pressure signal, conveyed via propagation of pressure through the gas in the tubing, takes a larger amount of time to reach the ventilator (or the defibrillator or other device(s), which may be in communicative contact with the ventilator), causing a larger delay in what the processor of the ventilator 570 (and/or defibrillator or other connected device(s)) treats as the current patient airway pressure, relative to the actual current patient airway pressure, which time delay is represented as T2, where T2 is greater than T1. As such, using a sensing device results in less time delay. While other system factors can affect latency, distance of the sensing from the patient airway can be a major factor.

In some embodiments, for example, a system using a sensing device can lead to associated pressure sensing latency times of, e.g., 1-5 ms, 1-2 ms, 1 ms, 0.5-1 ms or 0.1-0.5 ms. By contrast, if, for example, 6 feet of tubing were used to reach from the patient airway to another sensing device (e.g., ventilator or defibrillator), that would cause approximately 6 ms of latency time associated with one way travel of the pressure signal at the speed of sound. Furthermore, if the gas flow source is located distant from the patient, this creates additional lag time in a changed flow reaching the patient. As such, in some embodiments, in addition to the use of a sensing device, a flow source may be located very close to the patient airway.

Additionally, in embodiments of a sensing device that includes an integrated mainstream CO2 sensor (as described in detail herein), the mainstream CO2 sensing can provide shorter lag times relative to, e.g., sidestream CO2 sensing via a sampling tube, where the CO2 sensing is less close to the patient airway. For example, sidestream CO2 sensing may cause several second, e.g., 3-4 second, lag, whereas mainstream CO2 sensing using a sensing device may only lead to lag of less than a second (e.g., 300-400 ms).

This additional delay (T2 minus T1), while on the order of milliseconds, can yet have significant negative consequences in connection with treatment, such as mechanical ventilation treatment. For example, patient ventilator asynchrony (PVA), which is a serious problem in mechanical ventilation, causing patient risk, may occur in situations in which inspiration or expiration is initiated by a mechanical ventilator out of synchronization with the timing of the inspiration and expiration reflected in the patient's breathing efforts (or other forms of PVA may be present). While lack of synchronization by a small enough amount of time may not cause PVA (for example, since a small enough amount of time in this regard will not result the patient's brain recognizing or resisting the lack of perfect synchronization with the patient's breathing efforts), lack of synchronization may trigger PVA. Lack of perfect synchronization in this regard can occur for many reasons. The additional delay (T2 minus T1) may cause lack of perfect synchronization or add to already imperfect synchronization, potentially causing the magnitude (e.g., in milliseconds) of the lack of synchronization to exceed a threshold that results in PVA, or may cause existing PVA to increase in magnitude. As such, in some embodiments, use of a sensing device with mechanical ventilation provides an advantage of reducing occurrence of, and/or reducing the magnitude of, PVA (e.g., reducing the amount of time difference between initiation of a patient's inspiratory or expiratory effort and initiation of inspiration or expiration by a mechanical ventilator).

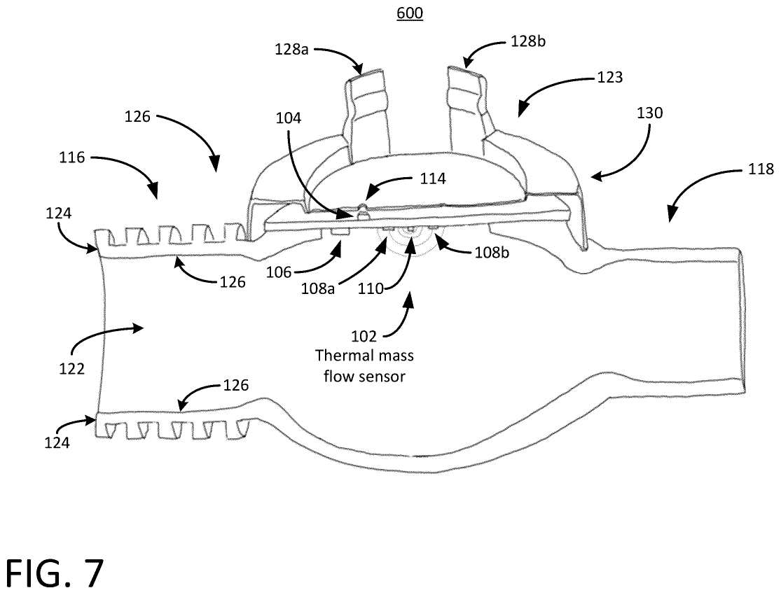

FIG. 7 is a cross-sectional view illustrating an example sensing device 600 including a hot wire anemometer based flow sensor 102, differential pressure sensing to sense internal pressure, and absolute external pressure sensing. The sensing device 600 may couple with a ventilation system, such as a BVM system, and with an airway of a patient, via a mask of the BVM system.

The sensing device 600 may include a gas flow conduit 124, where a tubular inside surface area 126 (or inside region, or lumen) of the conduit 124 defines a tubular gas flow region 122. On one end of the sensing device 600, a first connecting portion 116 may be for connection with a tube or passageway of the BVM system that couples with a patient airway (e.g., via a mask or endotracheal tube). The connection portion 116 may have an inner diameter that is suitably large for receiving an end of the tube or passageway of the BVM system, and may include a ribbed outer surface to form a male connection with a larger tube or passageway into which the connection portion 116 is inserted. On the other end of the sensing device 600, a second connecting portion 118, which may have a smaller inner diameter than the first connecting portion 116, may include a smooth outer surface to form a male connection with another tube or passageway of the BVM system, such as may allow coupling with a bag of the BVM system. The sensing device 600 further includes an upper connecting portion 123, which may be for connection by cable to another device, such as via a rotatable connection adapter, as described herein.

The sensing device 600 may include a housing 126 that includes the conduit 124. In some embodiments, the housing 126 may be integrated, e.g., not formed from separate but joined portions, such as by being a single formed portion of, e.g., plastic or polymer material (although, in various embodiments, it may be made from or include other materials), although, in other embodiments, the housing may include, e.g., multiple portions of plastic or polymer material that are joined or fixedly coupled together. As described herein, in some embodiments, the housing may be integrated and include features such as one or more flow conditioners, a CO2 sensor, and/or a rotatable connector. In the embodiment depicted, the sensing device 600 includes a top portion 130 with members, such as clasp members 128a, 128b, which may be used in attachment of a rotatable connection adapter, as described herein.

The hot wire anemometer based flow sensor 102 is disposed on or within the inside surface of the conduit 126, coupled with an electrical circuit board of the sensing device 600, to allow exposure to the gas flow through the inside 122 of the conduit 124, which may include inspiratory and expiratory gas flow. In some embodiments, a hot wire anemometer may include an electrical heating portion or heater that heats an electrically heated resistive portion, such as a wire 110, between two temperature sensors or thermocouples 108a-b, with one that senses the temperature of the gas surrounding the wire, and one that senses the temperature of the wire itself. In various embodiments, various types of hot wire anemometers, and their operation, may be used.

In some embodiments, flow rate is correlated with a temperature gradient between the two thermocouples 108a-b. Higher flow rate leads to a larger temperature gradient, since higher flow results in increased flow-based pushing of locally heated gas toward one of thermocouples (depending on flow direction) and away from the other.

In some embodiments, a hot wire anemometer may operate based on part on the principle that flowing gas in contact with the wire cools the wire in proportion with the rate of gas flow. The resistance of the wire changes based on the temperature of the wire. As such, a change in the resistance of the wire varies based on a change of the temperature of the wire. In various embodiments, various types of hot wire anemometers may be used, such as a constant current hot wire anemometer and/or a constant temperature hot wire anemometer. In a constant current hot wire anemometer, constant current is passed through the wire, and a temperature change causes a change in the resistance of the wire, which change in resistance is measured and used to measure flow rate of the gas. In a constant temperature hot wire anemometer, the temperature of the wire is kept constant by increasing the current passed through the wire to compensate for the cooling effect of the gas passing in contact with the wire, which increase in current is measured and used to measure a flow rate of the gas. Hot wire anemometers can provide very accurate flow rate measurements, even at very low flow rates.=

In the embodiment of FIG. 7, a differential pressure sensor 104 is used to measure the difference between the pressure inside 122 of the conduit 124 and the pressure outside of the conduit 125, which may be the ambient or environmental pressure. For example, in some embodiments, a differential pressure sensor may include two ends with a diaphragm between them. The diaphragm of the differential pressure sensor may be deflected in proportion to the difference in the two pressures, and measurement of the magnitude of deflection of the diaphragm may be used to measure the difference between the two pressures. In the embodiment of FIG. 6, the differential pressure sensor 104 includes one end 106 exposed to the pressure inside 122 of the conduit 124 and one end 104 exposed to the pressure outside of the conduit 124, to allow measurement of the difference between the two pressures. In addition, an absolute pressure sensor (not shown in FIG. 7) may be used to sense the pressure outside of the conduit 122. The pressure inside 122 of the conduit 124 can then be determined based on the measurement of the difference between the pressure inside 122 of the conduit 124 and the pressure outside of the conduit 124, along with the measurement of the absolute pressure outside of the conduit 124. In various embodiments, various types of differential and absolute pressure sensors may be used, such as a miniature electromechanical systems (MEMS) based pressure sensor, or a thermal flow sensor or hot wire anemometer based pressure sensor, among other types, which choices may be based, for example, on factors that may include, e.g., minimizing size and cost, or a balance between such factors. In some embodiments, a flow cytometer may be used, since it may be used in measurement and potentially analysis of CO2 and O2, but flow cytometers are expensive and heavy, and so, in some embodiments, are not used.

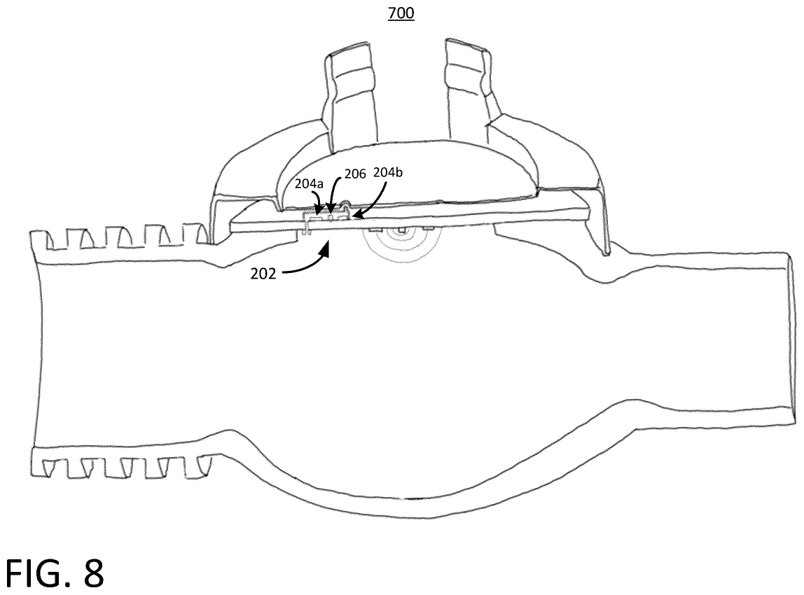

FIG. 8 is a cross-sectional view illustrating an example sensing device 700 including hot wire anemometer based flow sensing, hot wire anemometer based differential pressure sensing to sense internal pressure, and absolute external pressure sensing. In many ways, the sensing device 700 of FIG. 8 is similar to the sensing device 600 of FIG. 7. However, in the sensing device of FIG. 8, the differential pressure sensor 202, which senses the difference in pressure between the pressure inside 122 of the conduit 124 and the pressure outside of the conduit 125, is hot wire anemometer based, including temperature sensors 204a-b and resistive portion or wire 206. In various embodiments, while a hot wire anemometer based flow sensor may operate based on the principle that gas flow across the wire cools the wire in proportion to the rate of gas flow, a hot wire anemometer based pressure sensor may operate on the further principle that the rate of gas flow across the wire, which wire is disposed between regions of different pressures, varies in proportion to the difference in pressures between the regions. This follows from the fact that a difference in pressure between two regions causes a fluid, including a gas, to flow from the region of higher pressure to the region of lower pressure, as result of the pressure gradient force, where the rate of flow varies in proportion to the difference in the pressures.

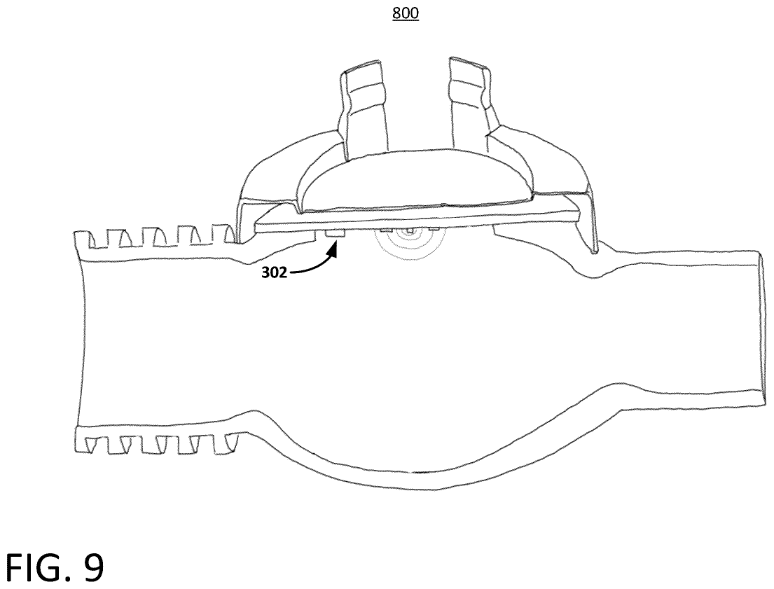

FIG. 9 is a cross-sectional view illustrating an example sensing device 800 including hot wire anemometer based flow sensing and absolute internal pressure sensing. The sensing device 800 shown in FIG. 9 is similar in many ways to the sensing devices 600, 700 shown in FIGS. 7 and 8. However, the sensing device 800 shown in FIG. 9 uses an absolute pressure sensor 302, disposed on, or in, the inside of the conduit 104, in measurement of the pressure inside 122 of the conduit. Since the absolute pressure sensor 302 is sufficient for measurement of the pressure inside 122 of the conduit 124, in some embodiments, no absolute pressure sensor is included to sense the pressure outside of the conduit 124. However, in some embodiments of the sensing device 800, an absolute pressure sensor to sense the pressure outside of the conduit 125 is none the less included, such as to allow correction or calibration of flow rate or pressure measurements inside 122 of the conduit 124 at altitude, where the ambient pressure may be sufficiently less than ambient pressure at sea level so as to affect such flow rate or pressure measurements measurably or significantly enough to call for correction or calibration accordingly.

In some embodiments, absolute pressure sensing outside of the conduit of a sensing device, since it allows for measurement of ambient pressure at all times, which can be used, e.g., in correcting for ambient pressure at altitude, as described herein. In some embodiments, however, ambient pressure may be determined sensed at another proximate deice (e.g. a ventilator or a defibrillator). In some embodiments, however, differential pressure sensing to sense the pressure inside of the conduit can be used to determine ambient pressure, since ambient pressure is reflected by the measured pressure when flow is zero. In some embodiments, including, in a sensing device, sensing of absolute pressure inside and outside of the conduit can be used in determining if BAP is being applied.

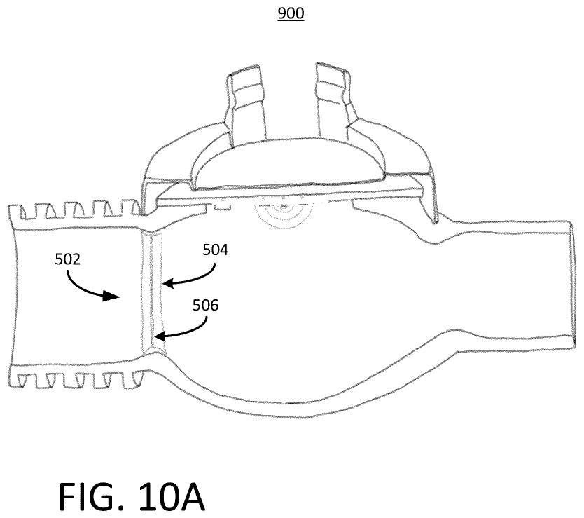

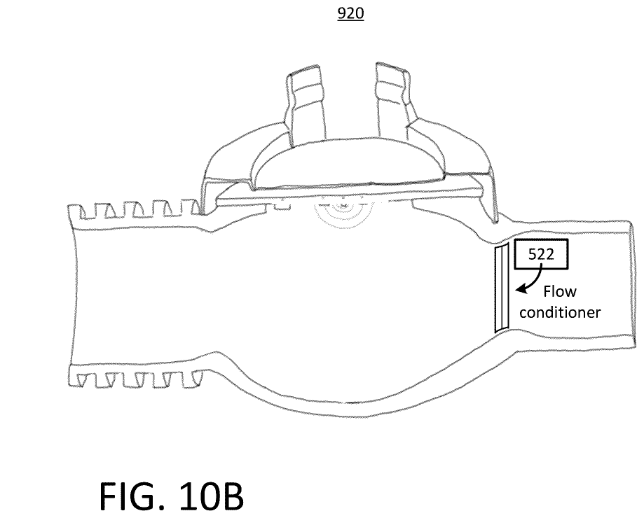

FIGS. 10A and 10B are cross-sectional views illustrating an example sensing devices 900, 920 including hot wire anemometer based flow sensing, absolute internal sensing, and a flow conditioner. The sensing devices shown in FIGS. 10A and 10B are similar in many ways to the sensing device 800 shown in FIG. 9, but include an added flow conditioner 502, 522. In the embodiment shown in FIG. 10A, a flow conditioner 502 is included on the side of the device closer to the patient airway, whereas, in FIG. 9B, a flow conditioner 522 is included on the side of the device further from the patient airway. Furthermore, in some embodiments, one or more flow conditioners may be included on both sides of a sensing device. In some embodiments, a flow conditioner may be included on the side further from the patient airway because there may be, e.g., less jetting from flow coming from the patient relative to flow going to the patient, which, in some embodiments, may result in a greater need or benefit from including a flow conditioner in the side further from the patient airway, relative to the side closer to the patient airway. In some embodiments, including a flow conditioner on both sides may provide advantages, such as improved flow conditioning and improved flow laminarization, but may lead to disadvantages such as increased cost, and increased design and assembly complexity and variability.

In some embodiments, one or more flow conditioners are included in or with a sensing device. Flow conditioners, including flow conditioners based on restrictors, screens, baffles and other particular flow obstructers, may improve accuracy and/or reliability of measurements of flow and pressure in the gas flow conduit of the sensing device. In fluid flows, lack of uniformity in regions of the flow, such as in rate and direction of flow, can reduce the accuracy of measurements of the flow rate and pressure in such regions. In some embodiments, a flow conditioner may increase flow and/or pressure measurement accuracy by, e.g., improving laminar flow of gas inside of the conduit. In some embodiments, a flow conditioner may condition or fully condition the flow, such as by causing the flow at a measurement site to be fully developed, e.g., no or low variability in the flow front in an axial direction and no or low changes to the flow front as it continues to travel.

In the embodiments shown in FIGS. 10A and 10B, the flow conditioners 502, 522 are not integrated with or formed as part of the housing 126 of the sensing device 900, but may be, e.g., joined or fixedly connected to the housing 126. In embodiment shown in FIG. 10A, the flow conditioner 502 includes outer portion 504 and an inner portions 506.

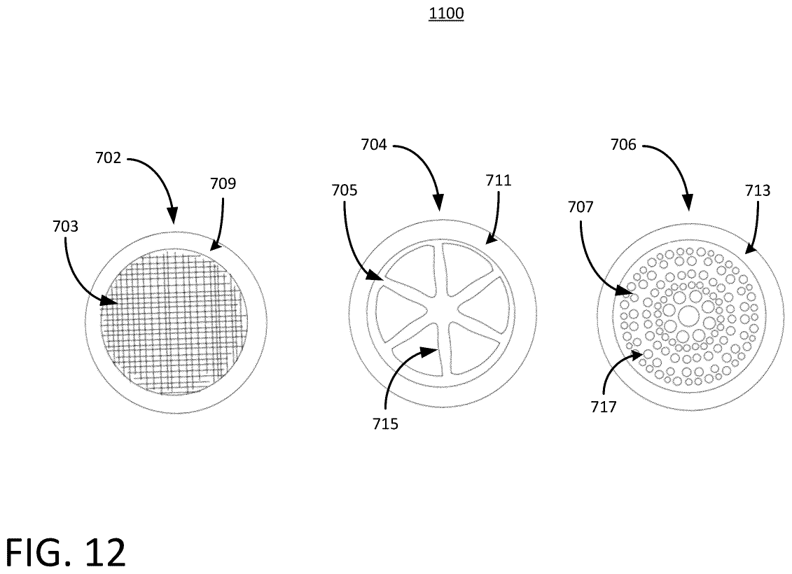

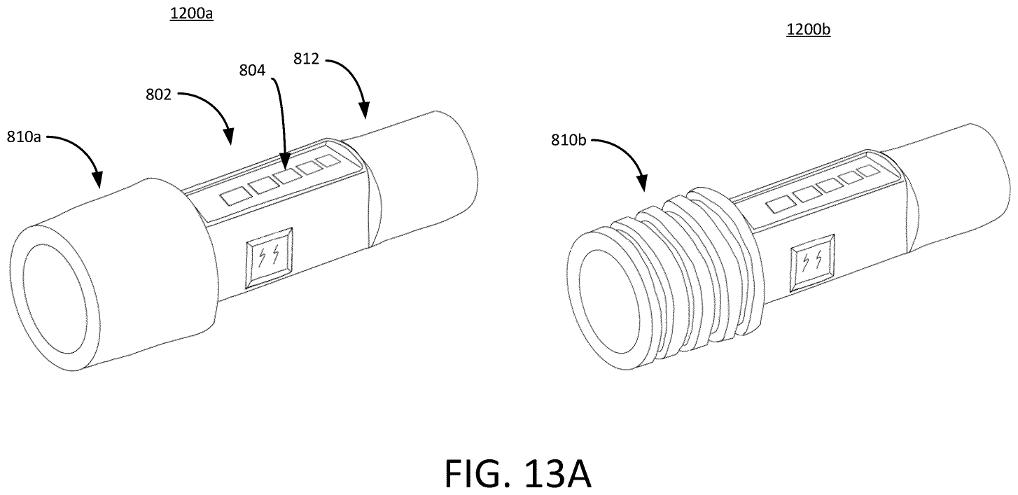

In some embodiments, the outer portion 504 may fit to, or be compressible and fit to, the inside surface that it meets, and may serve to hold the inner portion 506, which may, e.g., be a thin screen, screen like and/or film portion, which may have varying designs, examples of which are provided in FIG. 12. In some embodiments, the inner portion 506 is made as thin as practical, to help reduce any dead space and to assure that it does not interfere with the measurement site. However, in some embodiments, the inner portion 506 may be made thicker, which may improve flow conditioning performance, such as to better develop and/or laminarize the flow, but may, on the other hand, be more difficult to fit within the conduit or may interfere with the measurement site. In some embodiments, the thickness of the inner portion 506 may be selected based on factors that include these, as well as potentially including cost.

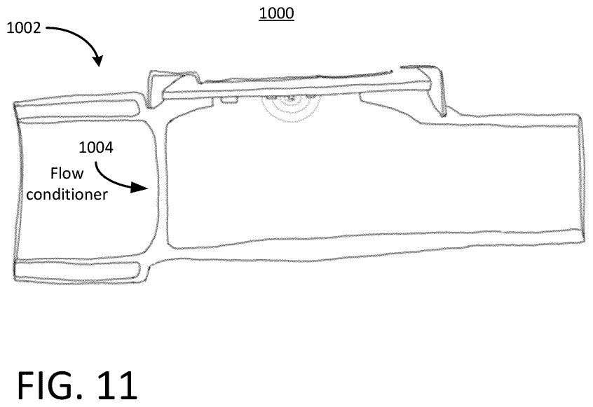

FIG. 11 is a cross-sectional view illustrating an example sensing device 1000 that is in many ways similar to the sensing device 900 of FIG. 10A. However, in the sensing device 1000 of FIG. 11, the flow conditioner 1004 is an integrated portion of the housing 1002 of the device, e.g., the housing 1002 may be a single formed material portion that includes the flow conditioner 1004 as a portion of the housing 1002. Additionally, in the embodiment depicted in FIG. 10, the sensing device 1000 does not include a top portion for allowing connection of a rotatable adapter.

In some embodiments, including one or more flow conditioners as a portion of the housing of the sensing, such as molded as part of the housing, device provides advantages such as decreased cost, and decreased assembly complexity and variability. However, including one or more flow conditioners as a portion of the housing of the sensing device may lead to disadvantages such as decreased moldability of, and decreased access to, the housing, as well as material selection. For example, it may be needed or desired that the inner portion of a flow conditioner be made of a different material than the housing of the sensing device, such as to allow accurate, detailed features and patterning of the inner portion. In that case, a required insert molding process may be made more difficult and complicated.

FIG. 12 includes views illustrating several different example types 702, 704, 706 of flow conditioners, which, in various embodiments, may be included in any of the sensing devices shown in previous figures, whether or not integrated with the housings thereof. In particular, in some embodiments, the example types 702, 704, 706 may be types of inner portions of flow conditioners. In various embodiments, various different types of flow conditioners may be used, including various shapes, configurations and patterns. For each of the example types 702, 704, 706 of flow conditioner inner portions, the associated view shows an upper surface 703, 705, 707 of each, where gas flow (inspiratory or expiratory) could pass into an outer surface and out of an inner surface (not shown), which inner surface, in various embodiments, may or may not be identical to the outer surface, depending the configuration of the flow conditioner along its depth (that is, along an axis generally parallel to gas flow through the associated gas flow conduit). The example types 702, 704, 706 include a circular outer surface, but, in other embodiments, other outer and inner surface shapes may be used, which may depend on the shape of the gas flow conduit in which, or as part of which, they are used. In various embodiments, the shape of a flow conditioner may not vary throughout its depth, or may vary in different ways (e.g., with uniform, or increasingly smaller or larger openings, from an outer surface to an inner surface). In the embodiments shown, the depth of each of the flow conditioner inner portions 702, 704, 706 is much less than the diameter, but various embodiments include various depths and proportions of depth to surface diameter or other end to end surface dimension, for example. In some embodiments, factors that may be taken into account in flow conditioner inner portions may include manufacturability and cost, which may be higher for some more complex designs and patterns. Furthermore, in some embodiments, thinner flow conditioner inner portions with smaller holes may provide improved flow conditioning, but may require a more complex or expensive design, such as by requiring an insert in a mold or a separate part of the assembly.

As depicted, each of the flow conditioners 702, 704, 706 includes an outer surface 703, 705, 707 including an outer ring 709, 711, 713 without openings (which, in other embodiments, may differ or not be included), and an area 709, 711, 713 inside of the outer ring that includes: for surface 703, a screen type surface pattern; for surface 705, a spoked 715 wheel type pattern; and, for surface 707, a pattern including circular openings 717 of various sizes, placed in generally circular rings along surface 707, including spacing between some of the circular openings 717 along some of the rings. However, in various embodiments, a variety of different configurations, both along the outer and inner surfaces, and along depths of flow conditioners, may be used. In some embodiments, such configurations may be determined so as to be optimized for the particular use, such as may include taking into account various factors, e.g., the type of sensing device, the shape and configuration of the gas flow conduit, the placement and position (e.g. angle) of the flow conditioner within the conduit, and the type and placement of the flow and pressure sensors within the conduit. Furthermore, in some embodiments, flow conditioner selection and placement may be selected to achieve a particular less conditioned flow front profile that is not fully developed but that focuses the flow front to a particular cross-sectional area to direct peak flow to the measurement site.

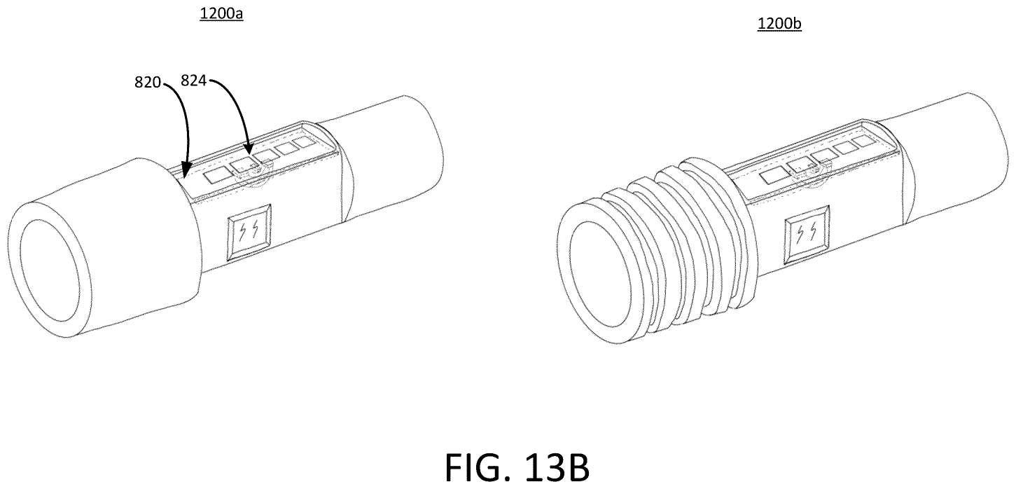

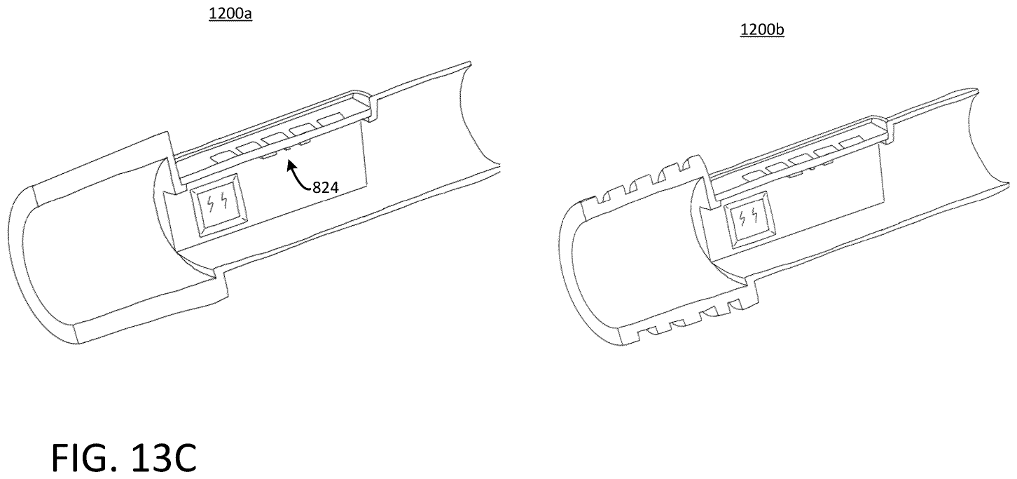

FIGS. 13A-C include a perspective view, an inside view and a cut-out view, respectively, illustrating a sensing device 1200a, 1200b including integrated CO2 sensing, such as for use in capnography and measurement of EtCO2, and using hot wire anemometer based flow sensing. Embodiment 1200a includes an unribbed connector end 810a, while embodiment 1200b includes a ribbed connector end 810b. In particular, as shown in FIG. 13A, the sensing device includes a mainstream, wrap-around mainstream CO2 sensor 802, which may essentially at least in part wrap around a central portion of the gas flow conduit of the sensing device. As shown in FIG. 13A, the CO2 sensor 802 includes multiple electrical contacts 804. In the inside view of FIG. 13B and cut-out view of FIG. 13C, an interior hot wire anemometer flow sensor 824, which may sense gas flow through the gas flow conduit of the sensing device, can be seen, coupled with an electrical circuit board 820 of the sensing device. In various embodiments, the CO2 sensor may be an integrated portion of a sensing device and/or of a housing of a sensing device, or may be capable of being coupled, or capable or being removably coupled, to a sensing device.

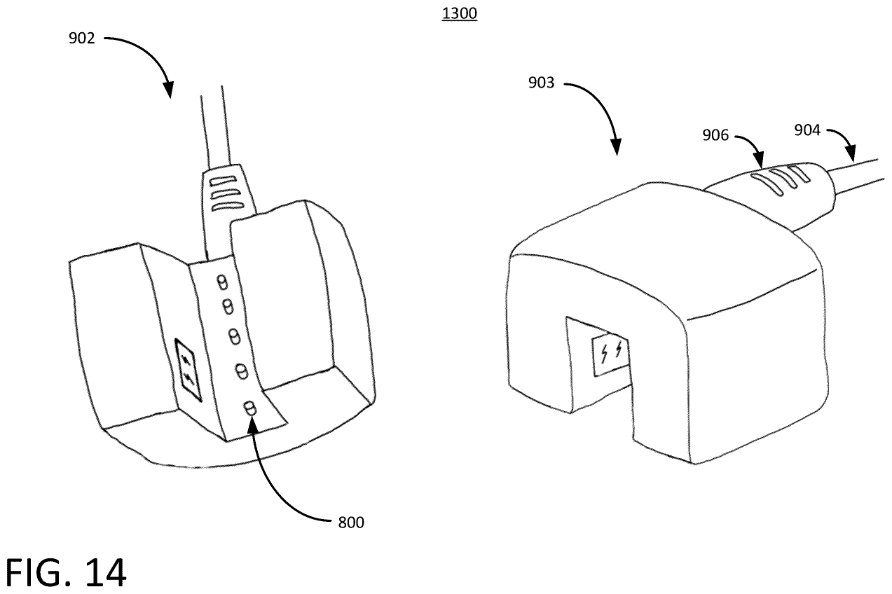

FIG. 14 includes bottom 902 and top 903 perspective views illustrating a CO2 sensor connection adapter 1300 for a sensing device including integrated CO2 sensing, such as the sensing device 1200 as depicted in FIGS. 13A-C. The connection adapter 1300 includes electrical contact pins 800 that may, for example, couple with the electrical contacts 804 of the sensing device 1200 as shown in FIGS. 13A-C, including for communication of data, such from, or to and from, the sensing device to one or more other connected devices. The connection adapter 1300 incudes, or connects with, a cable 904 for such connection, and may additionally include a strain relief portion 906 that surrounds and protects the cable 904.

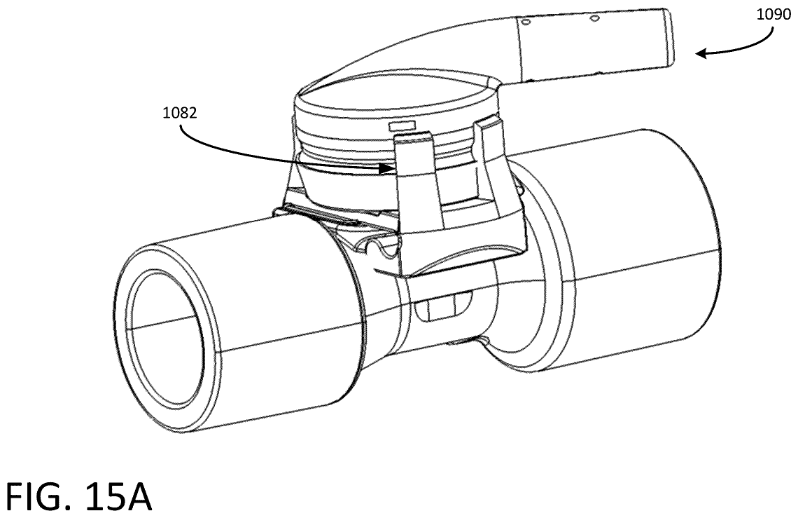

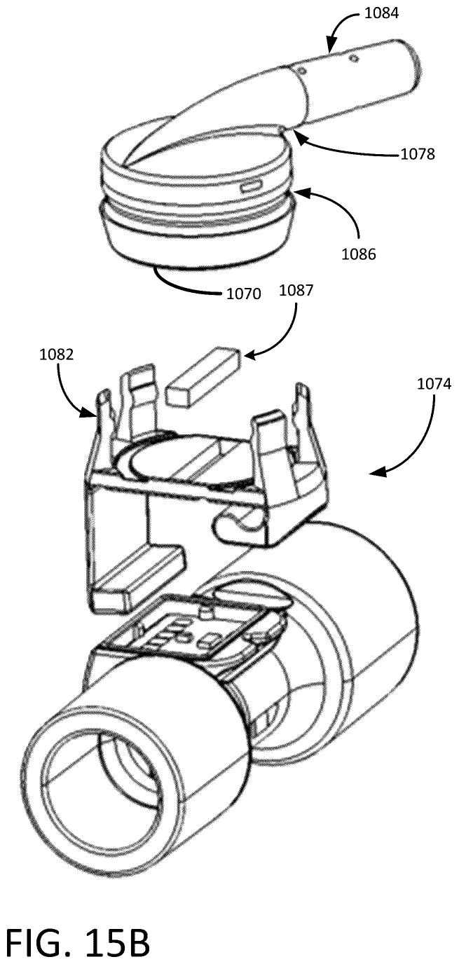

FIGS. 15A-B include a perspective view and an exploded view, respectively, illustrating a sensing device including, or attached to, a rotatable connection adapter 1090 with clasp attachment. Various features of embodiments of rotatable connection and a rotatable connection adapter, as described herein, may incorporate or utilize aspects of features described in previously incorporated by reference U.S. Pat. No. 11,433,211, issued on Sep. 6, 2022 and entitled, “Flow Sensor for Ventilation.”

In the embodiment depicted, the sensing device includes four clasp members 1082 (although, in other embodiments, other connecting members or features, and/or numbers thereof, may be used) to allow fixed attachment of the rotatable connection adapter 1090, where the clasp members 1082 may fit into a grooved portion 1086, as shown in FIG. 15B, of the outer surface of the rotatable connection adapter 1090. In various embodiments, the rotatable connection adapter 1090 may be removably attachable to the sensing device, or may be an integrated part of the sensing device or the housing of the sensing device. As depicted in FIG. 15B, the rotatable connection adapter 1090 includes a lower portion 1074 and an upper portion 1086, where the lower portion 1074 fixedly attaches to the sensing device, while the upper portion 1086 is rotatably connected to the lower portion 1074, and, in some embodiments, may be fully rotatable (e.g., through any of 360 degrees of rotation), such as in one or both directions of rotation. The upper portion 1086 may include a tubular strain relief member 1084 that surrounds and protects a cable that allows connection to another device. In some embodiments, the rotatable connection adapter 1090 may allow or help allow fast, convenient, reliable, simple connection of the sensing device to another device, which can be particularly important in emergency care situations.

The sensing device and/or the rotatable connection adapter 1090 may include one or more display or GUI indicators that may serve, for example, to signal to or notify a care provider that the rotatable connection adapter is correctly and/or communicatively connected to the sensing device (or that it is not so connected). For example, in the embodiment depicted, the sensing device includes indicators such as one or more LED indicators, which may be, e.g., illuminated, or illuminated in a particular color, when attachment is correct or incorrect, and may be visible from many different angles or positions, for example.

As shown in FIG. 15B, the sensing device may include an adapter 1087 that electrically couples with a contact pad 1070 of the rotatable connection adapter 1090. The adapter 1087 and contact pad may be used in maintaining electronic communication along with the rotatable coupling of the rotatable connection adapter 1090. Various features of embodiments of such an arrangement, as described herein, may incorporate or utilize aspects of features described in previously incorporated by reference U.S. Pat. No. 11,433,211, issued on Sep. 6, 2022 and entitled, “Flow Sensor for Ventilation,” including features relating to the adapter described therein.

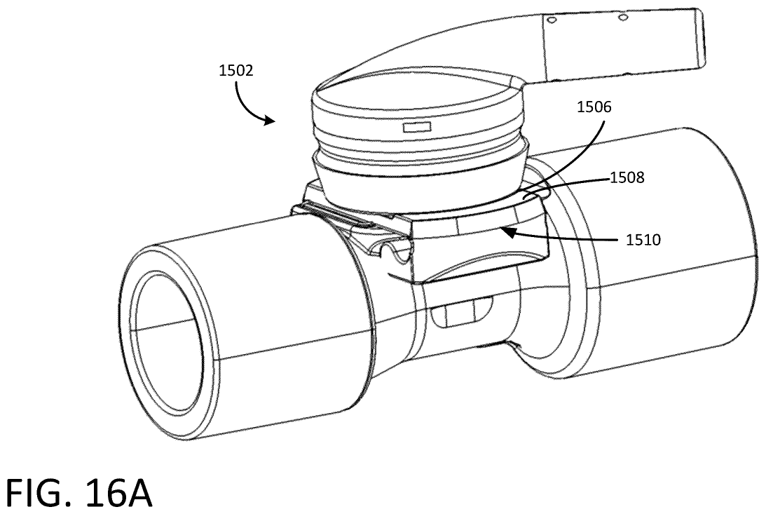

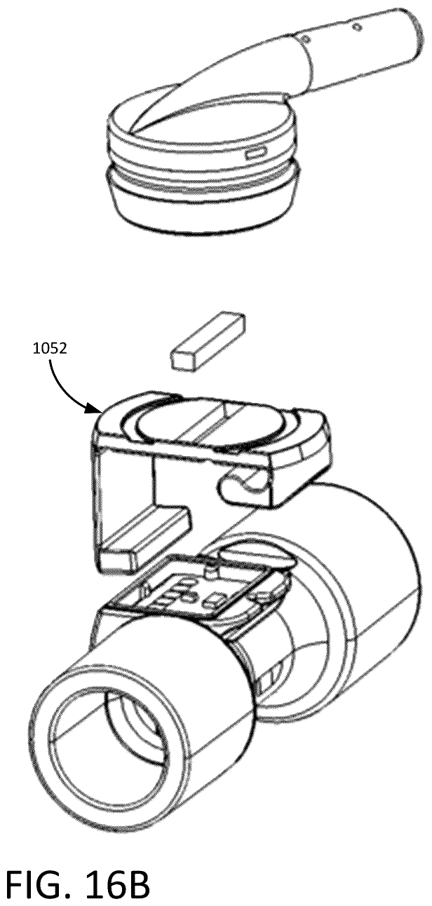

FIGS. 16A-B show an embodiment of a sensing device and rotatable connection adapter 1502 that is, in many ways, similar to those depicted in FIGS. 15A-B. However, in the embodiment depicted in FIGS. 16A-B, rather than clasp attachment, the rotatable connection adapter 1502 fixedly attaches to the sensing device via magnetic attachment. Particularly, as shown, the sensing device includes a magnetized surface 1508 for magnetic attachment to a surface 1506 of the rotatable connection adapter 1502 (although, in other embodiments, for example, the rotatable connection adapter 1502 may include the magnetized surface for attachment to the sensing device). For example, in some embodiments, the rotatable connection adapter 1502 may include a ring magnet and the sensing device may include a metal ring insert or ferrous contacts, or vice versa, though, some embodiments, the former may have an advantage of lower cost.

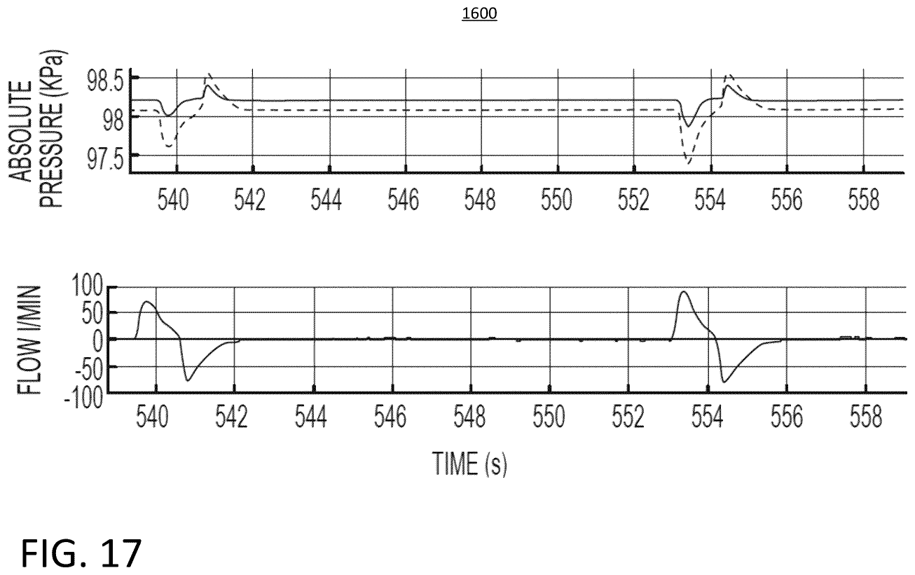

FIGS. 17-23 provide examples of data, waveforms and displays that can be determined or presented using signals from a sensing device according to some embodiments.

FIG. 17 depicts plots 1600 that show absolute pressure and flow rate for a spontaneously breathing patient over time, based on measurements using a sensing device. In a spontaneous breath, because air is drawn in due to pressure generated by downward movement of the diaphragm, the absolute pressure may indicate initial decrease during inspiration, followed by an increase in pressure during expiration.

In some cases, mere detection of a negative pressure may not be conclusive that spontaneously breathing is occurring; for example, incidental jostling or movement of nearby equipment may cause a negative pressure signal to be detected. Hence, to provide greater confidence of spontaneous breathing, it may be useful to set a minimum threshold of flow rate/volume (e.g., flow volume thresholds of at least 50 L, at least 100 mL, at least 150 L, etc. and/or flow rates of at least 5 L/min, at least 10 L/min, etc.), in combination with a negative pressure change. For example, when the system detects an initial negative pressure change and a threshold level of flow rate or volume, the system may provide an indication to the rescuer or other device that the patient may be undergoing spontaneous breathing. This information may be further useful in providing a rescuer with an indication that the patient may be experiencing return of spontaneous circulation (ROSC). For example, the system may provide a display to the rescuer of “Possible ROSC” and/or may provide appropriate instructions/guidance. To determine whether ROSC has actually occurred, other parameters may need to be considered, such as ETCO2, ECG signal, pulse detection, pulse oximetry, etc.

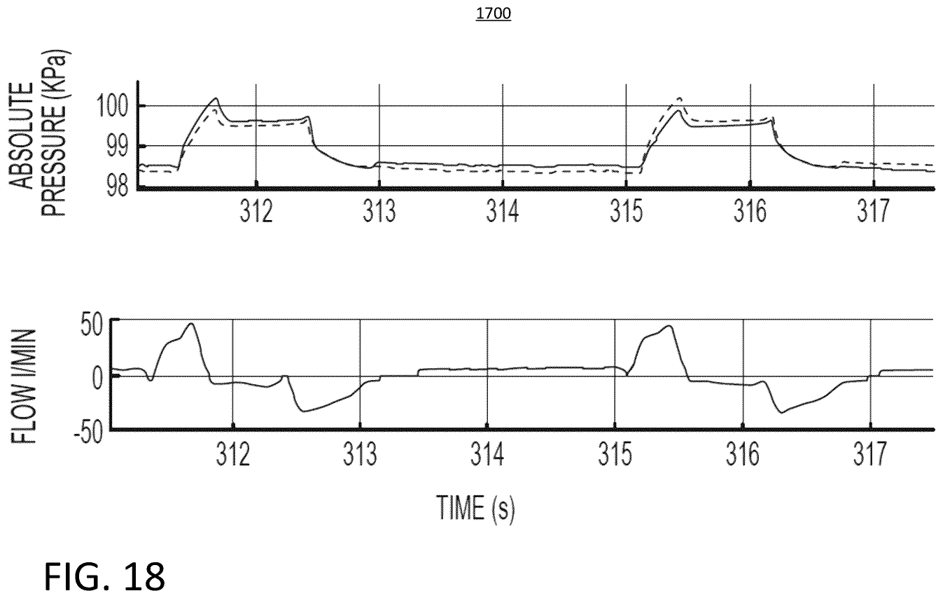

FIG. 18 depicts plots 1700 that show absolute pressure and flow rate over time for a patient receiving positive pressure ventilation with a mechanical ventilator over time, based on measurements using a sensing device. Here, both absolute pressure sensors are increased above atmospheric pressure for both inspiration and expiration. The pressure waveform is further characterized by a plateau at the height of the ventilator breath, which may be distinct from a positive pressure breath given by manual ventilation.

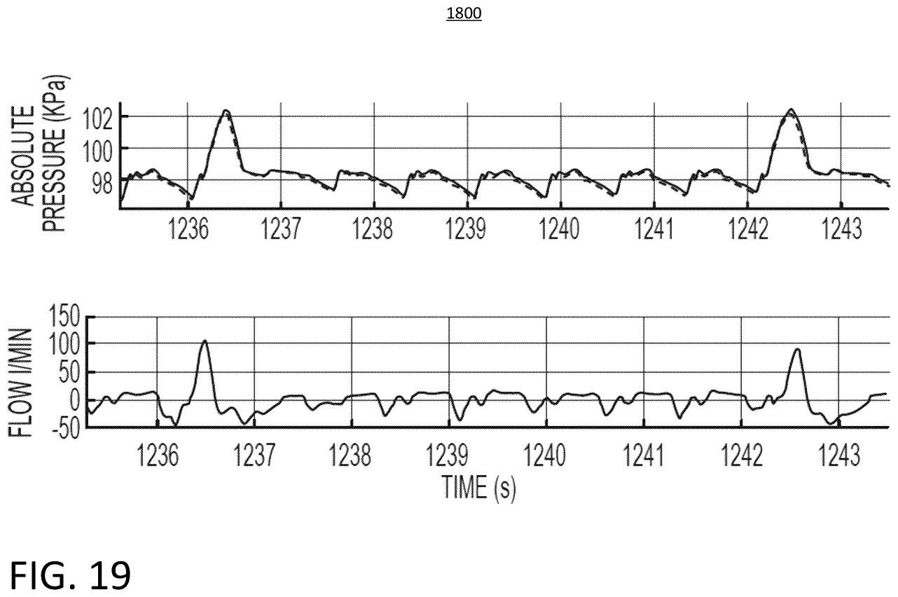

FIG. 19 depicts plots 1800 that show absolute pressure and flow rate over time for a patient receiving alternating BVM ventilation breaths and chest compressions, based on measurements using a sensing device. As shown, interposed between the two large manual ventilation peaks are a number of smaller peaks indicative of chest compressions. In BVM ventilation breaths, pressure increased above atmospheric pressure for both inspiration and expiration. In this respect, both manual and automated ventilation breaths may be distinguishable from spontaneous breaths in that they are positive pressure breaths. As such, flow and pressure waveform morphologies associated with the manually administered breath may be substantially different than that for a breath given by a typical automated ventilator. For example, the automated ventilator breath shows a more regular pressure and/or flow profile, including a plateau in pressure, which is not the case for the administered breath, which is more irregular in nature.

By determining the type of breath that is occurring, rescuers can be alerted whether the patient has begun spontaneous breathing and adjust the treatment protocol accordingly. For instance, for a spontaneously breathing patient, the rescuer may adjust how much additional ventilator support is necessary to give above what the patient is generating on their own. For example, when the patient is spontaneously breathing, the amount of pressure support assisted by manual or automated ventilation may be appropriately reduced. For example, the better the patient is able to breathe, the less pressure support may be required by positive pressure ventilation.

Further, as noted above, spontaneous breathing, while not fully conclusive, may be helpful evidence in determining whether the patient has achieved ROSC. Such information may be considered during an analysis of whether or not to administer a defibrillating shock to the patient. For example, if spontaneous breathing is detected, the rescuer or system may be triggered to perform a series of checks to determine if the patient has achieved or is likely to achieve ROSC. If ROSC has been achieved, then it may be decided that a shock should not be given. Such information may also be relevant for code review in evaluating whether rescuers were performing quality CPR. EMS rescuers are typically evaluated for how well they each performed CPR, whether or not ROSC has occurred. However, when ROSC has occurred in a patient, it may be determined that CPR may no longer be needed, hence, it may be preferable that EMS rescuers not be evaluated for their quality of CPR during ROSC. Thus, using techniques described herein, evidence of ROSC may be considered for determining whether EMS rescuers are evaluated for quality of CPR during the time period in which ROSC may have occurred. When ROSC is likely to have occurred, the code review may reflect that possibility and, in some cases, the score of EMS rescuers during the time in which ROSC may have occurred may be withheld from the overall evaluation.

Additionally, when a patient is experiencing spontaneous and/or agonal breathing, yet still receiving positive pressure breaths, the flow signal for the positive pressure breath may be inaccurate. Hence, when a spontaneous breath is detected at or around the time in which a positive pressure breath is administered, the flow parameters for that particular breath may be omitted or otherwise removed from the display or report provided to a user interface or other device associated with the resuscitative effort.

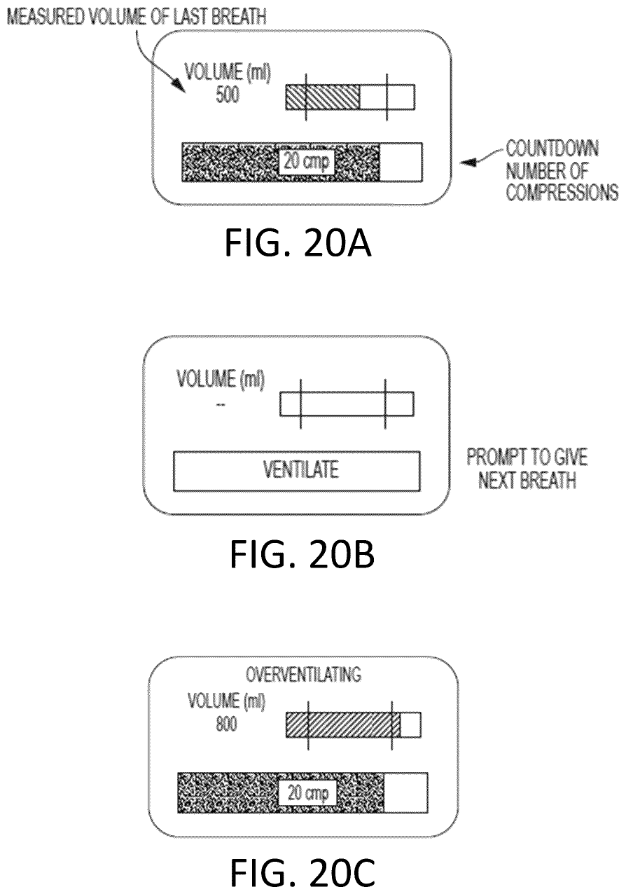

FIGS. 20A-C depict example display dashboards for providing feedback based on measurements using a sensing device. In some embodiments, a ventilation dashboard (e.g., of a display of a device) may display a number of parameters, such as indicia showing the flow rate and/or volume detected from a breath. The system may further determine what type of breath is being provided, for example, spontaneous or by positive pressure ventilation (e.g., manually given or automated breath). If ventilation breaths are not detected, the system determines that manual ventilation breaths are not being given to the patient, so then the system does not provide prompts to administer manual ventilation breaths. However, if ventilation breaths are detected, then the system determines whether the ventilation breaths are generated manually (e.g., from bag ventilation) or mechanically (e.g., from an automated ventilator). If the system determines that the breaths are being administered mechanically, then the system does not provide prompts for a user to administer manual ventilation breaths. However, if the system determines that the breaths are produced manually, then the system then continues to prompt the rescuer to administer breaths according to the appropriate treatment protocol.

In some embodiments, for patients who are able to breathe spontaneously, the system may be configured to cause positive pressure breaths to be synchronized with spontaneous breaths. It may be preferable for a positive pressure breath to be administered to a patient simultaneously during the beginning of an inspiratory breath, so that the gas more readily enters the lungs, for example, in contrast to expiratory flow during exhalation. Accordingly, when manual ventilations are detected, the system may sense when the patient is just beginning a spontaneous inspiratory breath, and immediately prompt the rescuer to administer a manual ventilation so that the positive pressure breath is provided as air is being pulled into the lungs. This breath synchronization protocol may be provided as a mode to the system, and requires vigilance on the part of the system and the rescuer to determine when a spontaneous breath is occurring. The system may further provide a notification to the rescuer of the effectiveness of the breath synchronization, particularly if the attempted breath synchronization is ineffective or even harmful to the patient. If the breath synchronization protocol is disabled, ventilation prompts may be provided according to a timed rate such as in the case of a continuous breath protocol, or ventilation prompts may be provided according to the number of chest compressions that have been administered. For example, based on the 30:2 protocol, the system may countdown the number of chest compression that have occurred and prompt the rescuer to vent when the countdown has finished.

As noted above, a number of treatment protocols may be employed for manually ventilating a patient, such as the 30:2 protocol, providing 2 positive pressure breaths for 30 chest compressions. In this protocol, the system may display a ventilation dashboard (e.g., within an overall CPR dashboard, which may further include a chest compression dashboard, or simply having separate dashboards for ventilation and chest compressions) that shows an indication of how many chest compressions are remaining before a ventilation is to be administered. For example, as shown in FIG. 20A, the dashboard may include a countdown bar that decrements with each detected chest compression. In this case, when a chest compression is detected, the number of compressions remaining decrements by 1 and the light-colored bar is slightly reduced in size. When the number of chest compressions reaches 0, or another value that provides an indication for a breath to be applied, the system prompts the user to ventilate, as shown in FIG. 20B. Or, in another embodiment, the dashboard may include a displayed number (e.g., number provided within a circled area), or other suitable indicator for counting down to ventilation prompt.

For this manual ventilation technique, the dashboard further shows the volume of air provided to the patient for a given bagged breath. The dashboard depicted in FIG. 20A shows a numerical value of the ventilation volume (shown as 500 mL) and a bar graph providing a visual indication of the ventilation volume. The bar graph includes hashmarks which indicate to the user the preferred range of volume per breath that the patient should receive. Any suitable upper and lower limits for this range may be chosen, depending on the desired volume to be administered to the patient (e.g., 400 mL, 500 mL, 600 mL). In another embodiment, a circular region fills based on the detected flow volume and rate, and changes color based on whether the volume and rate are or are not within desired limits. For example, if the circular region turns green, then flow volume and rate are within the prescribed range. If the circular region turns yellow or red, then the flow volume is above, or below the prescribed range. An incomplete or partial filling of the circular region may be an indication that the volume delivered is insufficient. In general, the ventilation breath should include a sufficient amount of air to the lung that supplies enough of a source of oxygen for gas exchange and circulation to the body. Conversely, the ventilation breath should not be excessive, otherwise lung damage may occur. The lower limit for the volume per breath given to the patient may be approximately 100 mL, approximately 200 mL, approximately 250 L, approximately 300 mL, approximately 350 L, approximately 400 mL, etc.; conversely, the upper limit for the volume per breath given to the patient may be approximately 1500 mL, approximately 1200 mL, approximately 1000 mL, approximately 900 mL, approximately 800 mL, approximately 750 mL, approximately 700 mL, approximately 650 L, approximately 600 mL, etc. The desired ventilation volume may depend on patient characteristics, such as patient size, condition, age, weight, lung capacity, amongst others.

As noted above, the ventilation feedback provided may be customized for the particular victim, or alternatively may follow a set (default) protocol that does not differ from victim to victim. For example, the rate and volume of ventilation to provide a victim may depend on how long the victim has been suffering from a current condition. Thus, a rescuer may try to ascertain how long the victim has been down, or a time stamp from the time at which an emergency was called in may be used as a proxy. Also, various states of the victim may be relevant to the treatment protocol (e.g., rate and volume of ventilation) to be provided to the victim, including, for example, whether the patient is pediatric or adult; patient condition (e.g. traumatic brain injury, cerebral herniation, cardiac arrest); ECG characteristics that suggest different ventilation requirements (e.g., patients with ventricular fibrillation may have lower ventilation requirements than patients with asystole or PEA; etiology of disease (e.g., cardiac arrest due to drowning vs. presumed myocardial infarction; duration of patient downtime for cardiac arrest; presence/absence of (effective) bystander CPR (compressions and/or ventilations) prior to arrival of EMS; ETCO2 levels (e.g., recommendations to titrate ventilation rate to achieve a particular end tidal CO2 value; SpO2 levels (e.g., adjust ventilation rate to achieve optimal peripheral oxygen saturation); and impact of SmO2 (muscle oxygenation) and/or tissue PH levels. Depending on what input parameters are provided to the system, an appropriate treatment protocol may be selected and/or adjusted, and ultimately communicated to the user. Though, in certain situations, such as those where the patient condition is rapidly deteriorating (e.g., sudden oxygen desaturation, patient becomes extremely hypotensive, etc.) it may be preferable that any changes in protocol not be displayed or otherwise communicated to the user, e.g., it may be better to maintain the current the current target ventilation parameters, rather than adjusting them.

When a positive pressure breath is provided, the bar graph on the dashboard may fill so as to show the instantaneous volume of air provided to the patient. Hence, a manual bagger may view the bar graph to determine whether the total volume administered to the patient is within desired limits. As shown in FIG. 20A, the 500 mL ventilation volume falls within the specified limits. As also shown, when the positive pressure breath is completed and chest compressions are to be administered, the ventilation dashboard may continue to show information regarding the previously provided ventilation breath during the current set of chest compressions, until the next ventilation breath is administered. The user should then provide a positive pressure breath similar to the previous breath. As further shown in FIG. 20B, when the dashboard prompts the user to ventilate, the volume indication (numerical value and bar graph) resets and provides the ventilation volume when the breath is applied. FIG. 20C shows the ventilation volume to be 800 mL, which falls outside of the specified range. Accordingly, the dashboard provides an indication to the user that the patient has been overventilated. This may provide a signal to the user to lessen the ventilation volume of the next positive pressure breath.

FIG. 21 depicts an example ventilation timer display based on measurements using a sensing device. The ventilation timer 500 provides information to the rescuer to help the rescuer control the rate of ventilation provided to the patient. The ventilation timer 500 can include a bar 506 (or other shape) that fills as time elapses between breaths. The bar 506 can include scaling information (e.g., tick marks on the graph) that provide information about the elapsed time 502 and/or ventilation rate 504. The elapsed time 502 provides an indication of the amount of time that has passed since the last ventilation event and the ventilation rate 504 provides the number of breaths per minute (e.g., 5 seconds between breaths=12 breaths/minute).

The information displayed on the ventilation timer 500 is based on ventilation related data received from a device that detects when a ventilation has been delivered (e.g., a flow meter, capnography, thoracic impedance). The ventilation related information is used by a computer to provide an input indicating when to re-start the timer such that the elapsed time can be determined.

In some examples, the information presented on the ventilation timer 500 can be color coded or otherwise supplemented by a visual indicator of ranges that indicate adequate ventilation or poor or suboptimal ventilation. In one example, the color of the bar 506 in the ventilation timer can change based on the adequacy of the ventilation. For example, the bar could be colored green when proper ventilation is being provided and yellow or red when the ventilation falls outside the desired range of respiration rates. Additionally, in some examples, an indication of whether the user should increase or decrease the rate of respiration could be provided. Additionally, in some examples, an indication of the optimal elapsed time/ventilation rate could be provided such as by overlaying a line or other indicator at the desired level so the rescuer can attempt to have the bar 506 match the displayed optimal timing indicator.

In some additional examples, the information presented in the ventilation timer 500 can be color coded or otherwise supplemented by other visual indicator based on the nature of the underlying condition being treated, e.g. respiratory distress vs cardiac arrest vs TBI. Additionally, the range that is indicated as an optimal or an acceptable respiration rate can change based on information from one or more physiologic monitoring sensors and estimate from those sensor(s) of the underlying status of the patient's cardiopulmonary status. Such physiologic monitoring can be based, for example on information about EtCO2 (e.g., the partial pressure or maximal concentration of carbon dioxide, CO2 at the end of an exhaled breath, which is expressed as a percentage of CO2 or mmHg) and/or information about oxygen saturation from a pulse oximeter, a medical device that indirectly monitors the oxygen saturation of a patient's blood. Such physiologic monitoring can also include information from a tissue CO2 sensor that can be used to calculate the blood oxygen concentration, for example, based on the ventilation/perfusion ratio (or V/Q ratio) which provides a measurement used to assess the efficiency and adequacy of the matching of the amount air reaching the alveoli to the amount of blood reaching the alveoli (sometimes reported as the VQ mismatch which is used to express when the ventilation and the perfusion of a gas exchanging unit are not matched).

Minute volume CO2 measures may be helpful during ventilation because ETCO2 measures are dependent on the actual volume of gas delivered to the patient. The physiologic measure that the clinician may be titrating to may be the amount of CO2 gas exhaled from the patient, which is a helpful overall measure of the patient's physiologic state. Increasing the ventilation rate or tidal volume will cause ETCO2 values to decrease for a fixed CO2 gas elimination rate from a patient; this is not the case for minute-volume CO2 measures. Thus, minute volume measures of CO2 may be a useful parameter in situations where ventilation is being delivered in a manual fashion with a rescuer squeezing a ventilation bag where both ventilation rates and tidal volumes have been shown in multiple studies to be completely uncontrolled in the clinical environment.

CO2 concentration as a function of expired volume may be plotted such as in the form of a single-breath CO2 analysis (SBCO2). The SBCO2 curve has three phases: phase 1 made up of non-alveolar gas, or ventilatory dead-space gas, that is essentially free of CO2; phase 2 that is a transition phase with a characteristic S-shape that contains some amount CO2; and phase 3 that is the alveolar gas bearing the predominant quantity of exhaled CO2. Because the x-axis of the SBCO2, or expirogram as it is sometimes called, has units of volume, calculations can be made to determine both alveolar as well as non-alveolar deadspace based on techniques known to those skilled in the art.

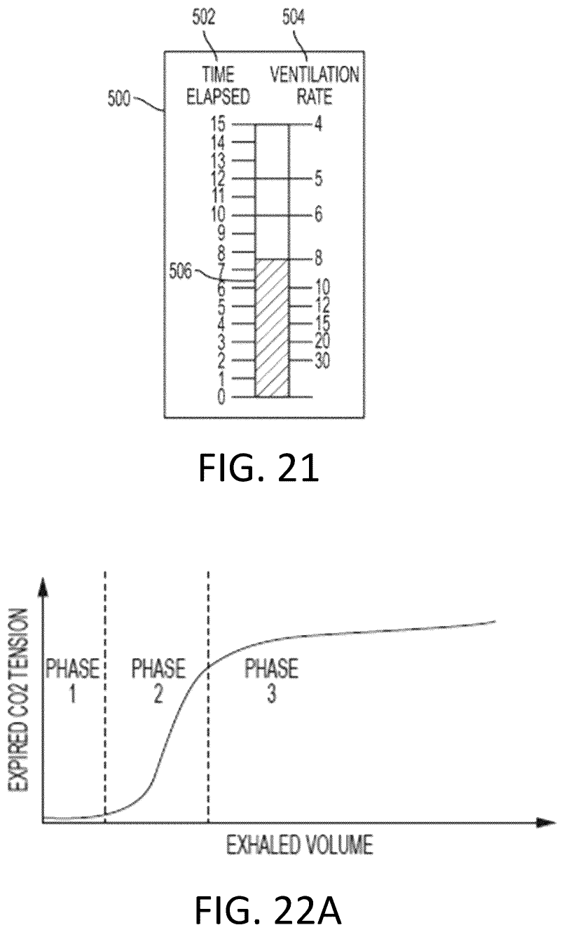

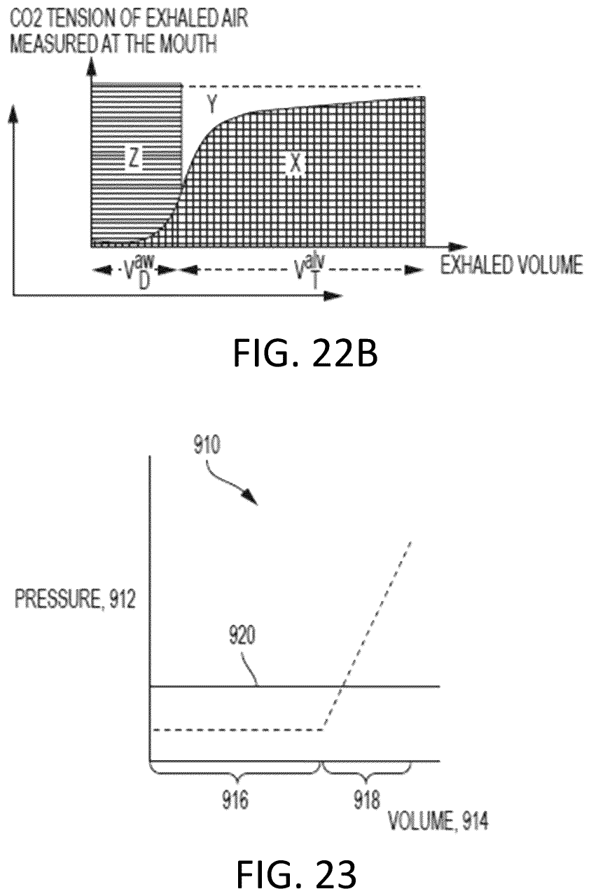

For example, FIGS. 22A and 22B each show the expired CO2 tension versus exhaled volume. The non-alveolar deadspace is the area of ‘Z’ in FIG. 22A and the alveolar deadspace is the area of ‘Y’. The sum of these two deadspaces does not produce any gas exchange in the patient, so this sets the minimum ventilation volume for each patient. Additionally, including dynamic lung compliance in the calculation of overall lung volume using SBCO2 curves may enhance the accuracy produced by SBCO2 based calculations.

FIG. 23 is an exemplary graph 910 of pressure 912 versus volume 914 during BVM ventilation. Indications of pressure versus volume can be used as a guide for determining an optimal tidal volume for manual ventilation of victims. In general, for adult patients and older children tidal volume (Vt) is calculated in milliliters per kilogram and values in the range of 6 to 8 mL/kg are often used. Hence a patient weighing 70 kg would get a Vt of 420-480 mL. However, in the field, a rescuer often will not have access to patient weight to calculate a desired tidal volume. Thus, it can be beneficial to provide feedback to the rescuer on an appropriate tidal volume without performing calculations based on patient specific weight or age parameters.

In some cases, the height may be used as an estimate for weight and/or size of the patient. For example, Broselow pediatric emergency tape may be used as such as estimator. Broselow tape, generally speaking, has a scale provided as a color-coded measure for pediatric emergencies. The Broselow scale relates the height of a child (up to approximately 12 years in age) as measured by the tape to his/her weight (up to approximately 36 kg or 80 lbs), which is useful to provide medical treatment instructions, such as medication dosages, the size of the equipment that should be used, the level of defibrillation shock voltage, amongst others. Particular to children is the need to calculate the relevant therapies for each child individually, primarily based on size. And in an emergency, the time required to make such calculation(s) may detract from valuable time needed to evaluate, initiate, and monitor patient treatment. A similar Broselow-type scale may be used to determine the ventilation parameters that would apply for pediatric or adult patients. That is, ventilation parameters may vary based on the size of the patient, whether pediatric or adult. For example, the estimated tidal volume, breath volume/rate, etc. may be provided as targets to the rescuer administering the therapy. Tidal volume may generally be determined based on predicted body weight (not necessarily actual body weight because overweight people generally have similarly sized lungs as thinner people of the same height), estimated from gender and height. For example, the predicted body weight (kg) for females is generally (50+2.3 (height (in)−60); and for males is generally (45.5+2.3 (height (in)−60). In some embodiments, the system may be configured to estimate size/weight of the patient and, hence, select an appropriate therapy based on an input (e.g., via a user interface) of patient height or other size information. The feedback information provided to the rescuer may then be adjusted based on the selected therapy.

In manual ventilation, as shown in FIG. 23, as the volume 914 of air administered to the victim increases, initially the pressure remains low and substantially constant (portion 916) as the lungs inflate. As the lungs near full inflation, the pressure required to administer additional volume is increased (portion 918). As the pressure rises above 45 cm H2O (4.4 kPa) for adults, the risk of barotrauma is increased and efforts should be made to try to reduce the peak airway pressure. In infants and children, even lower levels of peak pressure may cause damage. In general, keeping peak pressures below 30 cm H2O (2.9 kPa) (denoted by line 920) may be desirable. Thus, by observing changes in the peak pressure or by observing changes in pressure per changes in volume, a determination can be made of when a desirable tidal volume has been administered to the victim.

The change in volume divided by change in pressure is sometimes referred to as a compliance measurement. Compliance is a measure of the “stiffness” of the lung and chest wall. The mathematical formula for compliance (C) is change in volume divided by change in pressure. The higher the compliance, the more easily the lungs will inflate in response to positive pressure. Compliance values can be calculated and used to provide feedback on tidal volume to the rescuer.

The ventilation dashboard may be provided as a standalone display or as a portion of a CPR dashboard or larger display, for example, on the user interface display of a hospital or EMS monitor. For example, a general display may include a wide variety of physiological data of the patient, such as ECG, EtCO2, SpO2, blood pressure, muscle oxygenation, muscle pH, diagnostic information, heart rate, temperature, etc. The general display may also include a CPR dashboard, which may provide the user with resuscitative information useful for assisting a user in providing resuscitative treatment to the patient, for example, in maintaining the quality of CPR, including chest compressions and/or ventilations. Accordingly, the CPR dashboard may include a chest compression dashboard, for tracking parameters useful for providing quality chest compressions, such as depth, rate and/or release. The CPR dashboard may also include a ventilation dashboard, for tracking parameters useful for providing quality ventilations, such as ventilation rate, volume, minute volume and/or ventilation timing.

Other resuscitative information for assisting the user in providing resuscitative treatment may be provided, for example, based on a number of embodiments described herein. For instance, such resuscitative information may include feedback for instructing a user to adjust gas flow (e.g., flow rate, flow volume, minute volume) through the lumen, feedback for instructing a user to adjust placement of an intubation tube, alerts to a user and/or machine that overventilation has occurred or may occur, a countdown of the number of chest compressions until a subsequent ventilation is to be applied, a countdown of the time until a subsequent ventilation is to be applied, a number of chest compressions applied based on pressure and/or flow rate signals, a determination of whether a detected breath is due to spontaneous breathing, manually applied ventilation or automatically applied ventilation, instructions to the user to check the patient based on an indication of whether ROSC may have occurred, an indication of the determined peak inspiratory pressure, flow rate and/or volume of gas flowing through the lumen of the flow conduit, amongst others in accordance with embodiments discussed herein.

The physiological data of the patient and the resuscitative information may be provided on a display interface of a defibrillator and/or monitor. In some embodiments, the physiological data of the patient may be provided on a first portion of the display and the resuscitative information of assisting the user in providing resuscitative treatment may be provided on a second portion of the display. That is, depending on what information may be most relevant in treating the patient, the display may provide both physiological data of the patient and resuscitative information for assisting the user.

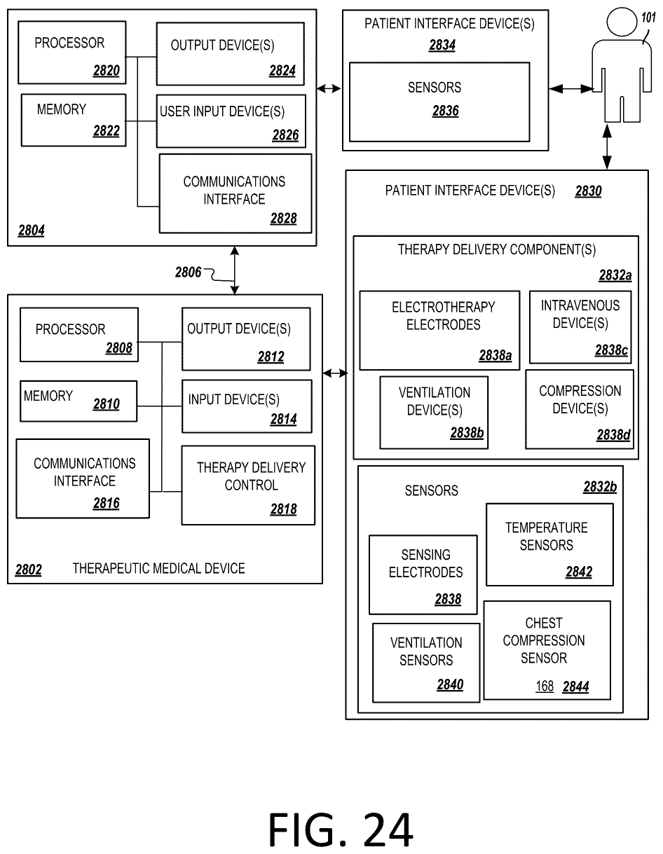

FIG. 24 illustrates an example of components of various devices described with reference to prior figures. The components 2808, 2810, 2812, 2814, 2816, and 2818 are communicatively coupled (directly and/or indirectly) to each other for bi-directional communication. Similarly, the components 2820, 2822, 2824, 2826, and 2828 are communicatively coupled (directly and/or indirectly) to each other for bi-directional communication.

In some implementations, the components 2808, 2810, 2816, and/or 2818 of the therapeutic medical device 2802 may be combined into one or more discrete components and components 2816 and/or 2818 may be part of the processor 2808. The processor 2808 and the memory 2810 may include and/or be coupled to associated circuitry in order to perform the functions described herein. Additionally, the components 2820, 2822, and 2828 of companion device 2804 may be combined into one or more discrete components and component 2828 may be part of the processor 2820. The processor 2820 and the memory 2822 may include and/or be coupled to associated circuitry in order to perform the functions described herein.

In some implementations, the therapeutic medical device 2802 may include the therapy delivery control module 2818. For example, the therapy delivery control module 2818 may be an electrotherapy delivery circuit that includes one or more high-voltage capacitors configured to store electrical energy for a pacing pulse or a defibrillating pulse. The electrotherapy delivery circuit may further include resistors, additional capacitors, relays and/or switches, electrical bridges such as an H-bridge (e.g., including a plurality of insulated gate bipolar transistors or IGBTs), voltage measuring components, and/or current measuring components. As another example, the therapy delivery control module 2818 may be a compression device electro-mechanical controller configured to control a mechanical compression device. As a further example, the therapy delivery control module 2818 may be an electro-mechanical controller configured to control drug delivery, temperature management, ventilation, and/or other type of therapy delivery.

The therapeutic medical device 2802 may incorporate and/or be configured to couple to one or more patient interface devices 2830 and patient interface devices (e.g., of or coupled with a ventilation system such as a BVM ventilation system) that may be coupled with a patient 2849. The patient interface devices 2830 may include one or more therapy delivery component(s) 2832a and one or more sensor(s) 2832b (some of which may be included as part of, e.g., a sensing device according to various embodiments described herein). Similarly, the companion device 2804 may be adapted for medical use and may incorporate and/or be configured to couple to one or more patient interface device(s) 2834. The patient interface device(s) 2834 may include one or more sensors 2836. The sensor(s) 2836 may be substantially as described herein with regard to the sensor(s) 2832b.

The sensor(s) 2832b and 2836 may include sensing electrodes (e.g., the sensing electrodes 2838), ventilation and/or respiration sensors (e.g., the ventilation and/or respiration sensors 2830), temperature sensors (e.g., the temperature sensor 2842), chest compression sensors (e.g., the chest compression sensor 2844), etc. In some implementations, the information obtained from the sensors 2832b and 2836 can be used to generate information displayed at the therapeutic medical device 2802 and simultaneously at the display views at companion device 2804 and described above. In one example, the sensing electrodes 2838 may include cardiac sensing electrodes. The cardiac sensing electrodes may be conductive and/or capacitive electrodes configured to measure changes in a patient's electrophysiology to measure the patient's ECG information. The sensing electrodes 2838 may further measure the transthoracic impedance and/or a heart rate of the patient. The ventilation and/or respiration sensors 2830 may include spirometry sensors, flow sensors, pressure sensors, oxygen and/or carbon dioxide sensors such as, for example, one or more of pulse oximetry sensors, oxygenation sensors (e.g., muscle oxygenation/pH), O2 gas sensors and capnography sensors, impedance sensors, and combinations thereof. The temperature sensors 2842 may include an infrared thermometer, a contact thermometer, a remote thermometer, a liquid crystal thermometer, a thermocouple, a thermistor, etc. and may measure patient temperature internally and/or externally. The chest compression sensor 2844 may include one or more motion sensors including, for example, one or more accelerometers, one or more force sensors, one or more magnetic sensors, one or more velocity sensors, one or more displacement sensors, etc. The chest compression sensor 2844 may provide one or more signals indicative of the chest motion to the therapeutic medical device 2802 via a wired and/or wireless connection. The chest compression sensor 2844 may be, for example, but not limited to, a compression puck, a smart-phone, a hand-held device, a wearable device, etc. The chest compression sensor 2844 may be configured to detect chest motion imparted by a rescuer and/or an automated chest compression device (e.g., a belt system, a piston system, etc.). The chest compression sensor 2844 may provide signals indicative of chest compression data including displacement data, velocity data, release velocity data, acceleration data, force data, compression rate data, dwell time data, hold time data, blood flow data, blood pressure data, etc. In an implementation, the defibrillation and/or pacing electrodes may include or be configured to couple to the chest compression sensor 2844.

In various implementations, the sensors 2832b and 2836 may include one or more sensor devices configured to provide sensor data that includes, for example, but not limited to ECG, blood pressure, heart rate, respiration rate, heart sounds, lung sounds, respiration sounds, end tidal CO₂, saturation of muscle oxygen (SMO₂), oxygen saturation (e.g., SpO₂ and/or PaO₂), cerebral blood flow, point of care laboratory measurements (e.g., lactate, glucose, etc.), temperature, electroencephalogram (EEG) signals, brain oxygen level, tissue pH, tissue fluid levels, images and/or videos via ultrasound, laryngoscopy, and/or other medical imaging techniques, near-infrared spectroscopy, pneumography, cardiography, and/or patient movement. Images and/or videos may be two-dimensional or three-dimensional, such a various forms of ultrasound imaging.

The one or more therapy delivery components 2832a may include electrotherapy electrodes (e.g., the electrotherapy electrodes 2838a), ventilation device(s) (e.g., the ventilation devices 2838b), intravenous device(s) (e.g., the intravenous devices 2838c), compression device(s) (e.g., the compression devices 2838d), etc. For example, the electrotherapy electrodes 2838a may include defibrillation electrodes, pacing electrodes, and combinations thereof. The ventilation devices 2838b may include a tube, a mask, an abdominal and/or chest compressor (e.g., a belt, a cuirass, etc.), etc. and combinations thereof. The intravenous devices 2838c may include drug delivery devices, fluid delivery devices, and combinations thereof. The compression devices 2838d may include mechanical compression devices such as abdominal compressors, chest compressors, belts, pistons, and combinations thereof. In various implementation, the therapy delivery component(s) 2832a may be configured to provide sensor data and/or be coupled to and/or incorporate sensors. For example, the electrotherapy electrodes 2838a may provide sensor data such as transthoracic impedance, ECG, heart rate, etc. Further the electrotherapy electrodes 2838a may include and or be coupled to a chest compression sensor. As another example, the ventilation devices 2838b may be coupled to and/or incorporate flow sensors, gas species sensors (e.g., oxygen sensor, carbon dioxide sensor, etc.), etc. As a further example, the intravenous devices 2838c may be coupled to and/or incorporate temperature sensors, flow sensors, blood pressure sensors, etc. As yet another example, the compression devices 2838d may be coupled to and/or incorporate chest compression sensors, patient position sensors, etc. The therapy delivery control modules 2818 may be configured to couple to and control the therapy delivery component(s) 2832a, respectively.

The one or more sensor(s) 2832b and 2836 and/or the therapy delivery component(s) 2832a may provide sensor data. The patient data provided at the display screens of the therapeutic medical device 2802 and companion device 2804 may display the sensor data. For example, the therapeutic medical device 2802 may process signals received from the sensor(s) 2832b and/or the therapy delivery component(s) 2832a to determine the sensor data. Similarly, the companion device 2804 may process signals received from the sensor(s) 2836 and/or sensor data from the sensors 2832b received via the therapeutic medical device 2802 to determine the sensor data.

The description set forth herein in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

As used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “substantially”, “about,” “proximate,” “minor variation,” and similar terms generally refer to ranges that include the identified value within a margin of 20%, 10%, 5%, or less than 5%, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

In some instances, variations of a term may be utilized that may refer to the same or similar concepts, and certain terms may have meanings that are informed by a particular context. Generally, sending, receiving, or transmitting of data may include by wired and/or wireless connection, and/or within one or more wired or wireless networks. Furthermore, sending from a first entity to a second entity, or to be received by the second entity, can include sending from the first entity to the second entity, or to be received by the second entity, directly from the first entity to the second entity, or indirectly via one or more intermediary entities.

While certain embodiments have been described, these embodiments have been presented by way of example only and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

1. A sensing device for use in ventilation treatment, comprising:
a gas flow conduit configured to be coupled with a patient airway;
a thermal mass flow sensor, disposed at least in part inside of the conduit, configured for measurement of a flow of gas inside of the conduit;
at least one pressure sensor configured for measurement of a pressure inside of the conduit, wherein the pressure inside of the conduit reflects a patient airway pressure; and
at least one flow conditioner configured to condition the flow of the gas inside of the conduit;
wherein the sensing device is configured to output signals for use in:
determining at least one waveform associated with a value of at least one ventilatory parameter over time; and
based at least in part on one or more morphological features of the at least one waveform, determine at least one current condition of the patient or at least one current condition relating to treatment of the patient.

2-7. (canceled)

8. The device of claim 1, wherein the device comprises at least one absolute pressure sensor configured to sense a pressure outside of the conduit.

9-10. (canceled)

11. The device of claim 1, wherein the device comprises at least one absolute pressure sensor configured to sense a pressure outside of the conduit, and wherein the at least one absolute pressure sensor is configured for use in calibration of the measurement of the flow of the gas inside of the conduit based on detection of an ambient pressure that is lower than a sea level ambient pressure, wherein the ambient pressure is at an altitude higher than sea level.

12. The device of claim 1, wherein the at least one pressure sensor comprises:
a differential pressure sensor, disposed at least in part inside of the conduit, configured to sense a pressure difference between a pressure inside of the conduit and a pressure outside of the conduit; and
an absolute pressure sensor configured to sense the pressure outside of the conduit,
wherein the differential pressure sensor and the absolute pressure sensor are configured for use in determination of the pressure inside of the conduit.

13-15. (canceled)

16. The device of claim 1, wherein the device is configured to provide sensed flow rate data relating to the flow of the gas inside of the conduit and sensed pressure data relating to the pressure inside of the conduit for use in determining ventilation feedback and chest compression feedback.

17-19. (canceled)

20. The device of claim 1, wherein the device is configured to provide sensed flow rate data and sensed pressure data for use in determining bag valve mask ventilation feedback, wherein the bag valve mask ventilation feedback relates to at least one of: a breath rate, a flow rate and a volume relating to the bag valve mask ventilation.

21-23. (canceled)

24. The device of claim 1, wherein the thermal mass flow sensor comprises a hot wire anemometer.

25-28. (canceled)

29. The device of claim 1, comprising a carbon dioxide sensor for sensing carbon dioxide in the gas inside of the conduit, wherein the conduit, the thermal mass flow sensor, the at least one pressure sensor and the carbon dioxide sensor are fixed relative to each other.

30-32. (canceled)

33. The device of claim 1, comprising a connection adapter configured for facilitating electrical connection of the device to a medical device, wherein the connection adapter is rotatable relative to the conduit.

34-35. (canceled)

36. The device of claim 1, wherein the measurement of the pressure inside of the conduit reflects a smaller delay, relative to a current patient airway pressure, than measurement of patient airway pressure using one or more sensors that are located at a distance from the patient's airway that is greater than a distance from the sensing device to the patient's airway, wherein the smaller delay causes a smaller risk of occurrence of patient ventilator asynchrony.

37-43. (canceled)

44. The device of claim 1, wherein the at least one ventilatory parameter comprises at least one concentration of at least one of: oxygen, carbon dioxide, nitrogen and nitric oxide.

45. The device of claim 1, wherein the at least one current condition of the patient comprises at least one of: spontaneous breathing, Return of spontaneous circulation (ROSC) and agonal breathing.

46. The device of claim 1, wherein the at least one current condition relating to treatment of the patient relates to at least one of: providing of bag valve mask (BVM) ventilation and providing of chest compressions.

47. The device of claim 1, wherein the at least one current condition relating to treatment of the patient relates to at least one of: providing of chest compressions with an active compression decompression (ACD) device, providing of manual chest compressions with an ACD device, providing of automated chest compressions with an ACD device, providing of chest compressions with an impedance threshold device (ITD), providing of chest compressions with an ACD device and an ITD, providing of conventional chest compressions, and providing of mechanical chest compressions without the use of an ITD or ACD device.

48. The device of claim 47, wherein the at least one morphological condition comprises at least one of: (a) at least one negative pressure associated with a patient airway pressure waveform, and (b) at least one flow rate associated with a patient airway flow rate waveform.

49. (canceled)

50. A system for use in ventilation treatment, comprising:
a gas flow conduit configured to be coupled with a patient airway;
a thermal mass flow sensor, disposed at least in part inside of the conduit, configured for measurement of a flow of gas inside of the conduit;
at least one pressure sensor configured for measurement of a pressure inside of the conduit, wherein the pressure inside of the conduit reflects a patient airway pressure; and
at least one processor configured to:
receive signals from the thermal mass flow sensor and the at least one pressure sensor; and
using the received signals, determine a flow rate of the gas inside of the conduit and a pressure inside of the conduit.

51-52. (canceled)

53. The system of claim 50, wherein the system comprises at least one absolute pressure sensor, and wherein the at least one processor is further configured to, using signals received from the at least one absolute pressure sensor, determine a pressure outside of the conduit.

54. The system of claim 53, wherein the at least one absolute pressure sensor is configured for use in calibration of the measurement of the flow of the gas inside of the conduit based on detection of an ambient pressure that is lower than a sea level ambient pressure.

55. The system of claim 50, wherein the at least one pressure sensor comprises:
a differential pressure sensor, disposed at least in part inside of the conduit, configured to sense a pressure difference between a pressure inside of the conduit and a pressure outside of the conduit; and
an absolute pressure sensor configured to sense the pressure outside of the conduit.

56-58. (canceled)

59. The system of claim 50, wherein the at least one processor is configured to use sensed flow rate data relating to the flow of the gas inside of the conduit and sensed pressure data relating to the pressure inside of the conduit in determining ventilation feedback and chest compression feedback.

60-66. (canceled)

67. The system of claim 50, wherein the thermal mass flow sensor comprises a hot wire anemometer.

68-69. (canceled)

70. The system of claim 50, comprising a carbon dioxide sensor for sensing carbon dioxide in the gas inside of the conduit, wherein the conduit, the thermal mass flow sensor, the at least one pressure sensor and the carbon dioxide sensor are fixed relative to each other.

71-73. (canceled)

74. The system of claim 50, comprising a connection adapter configured for facilitating electrical connection of the device to a medical device, wherein the connection adapter is rotatable relative to the conduit.

75-76. (canceled)

77. The system of claim 50, wherein the at least one processor is configured to, based at least in part on the determined flow rate of the gas inside of the conduit, detect a leak in the conduit.

78. The system of claim 50, wherein the at least one processor is configured to:
based at least in part on the determined pressure inside of the conduit, determine a patient airway pressure waveform reflecting the patient airway pressure over time; and
based at least in part on one or more morphological features of the patient airway pressure waveform, determine at least one current condition of the patient.

79. The system of claim 78, wherein the at least one current condition of the patient comprises at least one of: spontaneous breathing, ROSC and agonal breathing.

80-81. (canceled)

82. The system of claim 78, wherein the one or more morphological features comprise at least one of: a slope, a rise time, a fall time, a hold time, a plateau, a peak, a leading edge and a trailing edge.

83. The system of claim 78, wherein the at least one processor is configured to:
based at least in part on the determined flow of gas inside of the conduit, determine a flow waveform reflecting the flow over time; and
based at least in part on one or more morphological features of the flow waveform, determine the at least one current condition of the patient.

84. The system of claim 50, wherein the at least one processor is configured to:
based at least in part on the determined pressure inside of the conduit, determine a patient airway pressure waveform reflecting patient airway pressure over time; and
based at least in part on one or more morphological features of the waveform, determine at least one current condition relating to treatment.

85-94. (canceled)

95. The system of claim 50, wherein the system is configured to output signaling for use in:
determining at least one waveform associated with a value of at least one ventilatory parameter over time; and
based at least in part on one or more morphological features of the at least one waveform, determine at least one current condition of the patient or at least one current condition relating to treatment of the patient.

96-103. (canceled)

104. A method for providing feedback to a care provider in providing treatment to a patient, the method comprising:
using a thermal mass flow sensor disposed within a gas flow conduit of a sensing device used in providing ventilation treatment to the patient, the conduit being coupled with an airway of the patient, determining a flow rate of gas inside of the conduit;
using at least one pressure sensor disposed at least in part in the conduit, determining a pressure inside of the conduit, wherein the pressure inside of the conduit reflects a patient airway pressure;
based at least in part on the determined flow rate of the gas inside of the conduit and the determined pressure inside of the conduit, determining the feedback, comprising at least one of ventilation treatment feedback and chest compression feedback, to be provided to the care provider during the treatment of the patient; and
presenting the feedback to the care provider during the treatment of the patient.

105. The method of claim 104, wherein the treatment comprises the ventilation treatment, and wherein the feedback comprises ventilation treatment feedback.

106-107. (canceled)

108. The method of claim 104, wherein the treatment comprises chest compressions, and wherein the feedback comprises chest compression feedback.

109-134. (canceled)
